(19) United States
(12) Patent Application Publication
(10) Pub. No.: US 2014/0107113 A1
(43) Pub. Date: Apr. 17, 2014

(54) PHARMACEUTICAL COMPOSITION COMPRISING A TRPA1 ANTAGONIST AND A BETA-2 AGONIST

(71) Applicant: GLENMARK PHARMACEUTICALS SA, La Chaux-de-Fonds (CH)

(72) Inventors: Neelima Khairatkar-Joshi, Thane (W) (IN); Abhay Kulkarni, Navi Mumbai (IN); Dinesh Pradeep Wale, Osmanabad (IN); Anil Hari Kadam, Ahmednager (IN); Thamil Selvan Vaiyapuri, Trichy (IN)

(73) Assignee: GLENMARK PHARMACEUTICALS S.A., La Chaux-de-Fonds (CH)

(21) Appl. No.: 14/122,830

(22) PCT Filed: Jun. 21, 2012
(86) PCT No.: PCT/IB2012/053131

§ 371 Date: Nov. 27, 2013

(30) Foreign Application Priority Data
Jun. 22, 2011 (IN) 1811/MUM/2011

(51) Int. Cl. A61K 31/519; A61K 31/137; A61K 45/06; A61K 31/138; A61K 31/167

(52) U.S. Cl. CPC A61K 31/519; A61K 31/138; A61K 31/167; A61K 45/06; A61K 31/137
USPC 514/230.5; 514/260.1

(57) ABSTRACT

The present patent application relates to a pharmaceutical composition comprising a transient receptor potential ankyrin-1 receptor (“TRPA1”) antagonist and a beta-2 adrenergic receptor agonist (“beta-2 agonist”).

### PRIORITY DOCUMENT

This patent application claims priority to Indian Provisional Patent Application number 1811/MUM/2011 (filed on Jun. 22, 2011), the contents of which are incorporated by reference herein.

### TECHNICAL FIELD

The present patent application relates to a pharmaceutical composition comprising a transient receptor potential ankyrin-1 receptor (“TRPA1”) antagonist and a beta-2 adrenergic receptor agonist (“beta-2 agonist”). Particularly, the application provides a pharmaceutical composition comprising a TRPA1 antagonist having IC₅₀ for inhibiting human TRPA1 receptor activity of less than 1 micromolar with respect to TRPA1 activity and a beta-2 agonist; a process for preparing such composition; and its use in treating a respiratory disorder in a subject.

### BACKGROUND

Respiratory disorders related to airway inflammation include a number of severe lung diseases including asthma and chronic obstructive pulmonary disease (“COPD”). The airways of asthmatic patients are infiltrated by inflammatory leukocytes, of which the eosinophil is believed to be the most prominent component. Inflammatory sensitization of airway neurons is believed to increase nasal and cough sensitivity, heighten the sense of irritation, and promote fluid secretion, airway narrowing, and bronchoconstriction.

TRPA1 receptor activation in the airways by exogenous noxious stimuli, including cold temperatures (generally, less than about 17° C.), pungent natural compounds (e.g., mustard, cinnamon and garlic), tobacco smoke, tear gas and environmental irritants as well as by endogenous biochemical mediators released during inflammation, is supposed to be one of the mechanisms for neurogenic inflammation in the airways. Neurogenic inflammation is an important component of chronic airway diseases like COPD and asthma.

PCT Application Publication Nos. viz., WO 2004/055054, WO 2005/089206, WO 2007/073505, WO 2008/0949099, WO 2009/089082, WO 2009/002933 WO 2009/158719, WO 2009/144548, WO 2010/004390, WO 2010/109287, WO 2010/109334, WO 2010/109329, WO 2010/109328, WO 2010/125469 and WO 2010/004390 describe various transient receptor potential (“TRP”) receptor modulators.

Adrenergic receptors are believed to be the targets for catecholamines like adrenaline or noradrenaline. Beta-2 adrenergic receptors, a subtype of adrenergic receptors, are believed to be present in the cell membranes of various airway cells including the airway smooth muscle cell. The stimulation of the beta-2 adrenergic receptors in the airway cells is believed to lead to the relaxation of airway smooth muscle. In addition to the brochodilatory action, the beta-2 agonists are also believed to protect against bronchoconstrictor stimuli. Examples of the beta-2 agonists include salbutamol (INN: Albuterol), levosalbutamol, terbutaline, salmeterol, formoterol, metaproterenol, pirbuterol, bambuterol, procaterol, metaproterenol, fenoterol, bitolterol, indacaterol, vilanterol, arformoterol, olodaterol, abediterol, milveterol, bedoradrine, rirodrine clenbuterol, reproterol, PF-610355, GSK-597901, GSK-159802 and GSK-678007 or their salts.

Salmeterol xinafoate is chemically 4-hydroxy-a1-[[[6-(4-phenylbutoxy) hexyl]amino]methyl]-1,3-benzenedimethanol, 1-hydroxy-2-naphthalene carboxylate. Salmeterol xinafoate is commercially available as 50 mcg inhalation as SEREVENT® (marketed by Glaxo) in the United States. It is indicated for the maintenance treatment of asthma (as an add-on therapy, in the prevention of bronchospasm only as concomitant therapy with a long-term asthma control medication, such as an inhaled corticosteroid (ICS)), and prevention of exercise-induced bronchospasm.

Formoterol fumarate is chemically (±)-2-hydroxy-5-[(1RS)-1-hydroxy-2-[[(1RS)-2-(4-methoxyphenyl)-1methylethyl]-amino]ethyl]formanilide fumarate dehydrate. Formoterol fumarate is commercially available as and 12 mcg dry powder inhalation as FORADIL® (marketed by Novartis) in the United States. It is indicated for the treatment of asthma (as an add-on therapy) and in the prevention of bronchospasm, acute prevention of exercise-induced bronchospasm and for the long-term maintenance treatment of bronchoconstriction in patients with COPD including chronic bronchitis and emphysema.

Arformoterol is chemically N-[2-hydroxy-5-[(1R)-1-hydroxy-2-[[(2R)-1-(4-methoxyphenyl) propan-2-yl]amino]ethyl]phenyl]formamide. It is commercially available as 0.015 mg base/2 ml solution inhalation as BROVANA® (marketed by Sunovion) in the United States. It is indicated for the long-term, twice daily (morning and evening) maintenance treatment of bronchoconstriction in patients with chronic obstructive pulmonary disease (COPD), including chronic bronchitis and emphysema.

Indacaterol maleate is chemically (R)-5-[2-[(5,6-Diethyl-2,3-dihydro-1H-inden-2-yl)amino]-1-hydroxyethyl]-8-hydroxyquinolin-2(1H)-one. It is presently approved in Europe as Onbrez Breezhaler 150 mcg and 300 mcg capsule for inhalation (marketed by Novartis). It is indicated for maintenance bronchodilator treatment of airflow obstruction in adult patients with COPD.

Salbutamol sulphate (USAN: albuterol sulfate) is chemically, α1-[(tert-butylamino) methyl]-4-hydroxy-m-xylene-α, α′-diol sulphate (2:1)(salt). Salbutamol sulphate is commercially available as an inhalation aerosol, VENTOLIN HFA® (marketed by Glaxo SmithKline), as tablet (marketed by Mylan, among others), and as inhalation solution, ACCUNEB® (marketed by Dey) in the United States. It is indicated for the treatment and prevention of bronchospasm in patients with reversible obstructive airway disease and prevention of exercise-induced bronchospasm.

Levalbuterol (also called levosalbutamol) hydrochloride is chemically (R)-α1-[[(1,1-dimethylethyl)amino]methyl]-4-hydroxy-1,3-benzenedimethanol hydrochloride. It is commercially available in the USA as aerosol, metered inhalation equivalent 0.045 mg base/inh equivalent 0.0103% base; equivalent 0.021% base; equivalent 0.042% base; equivalent 0.25% base for administration by nebulization. It is indicated for the treatment or prevention of bronchospasm in adults, adolescents and children 6 years of age and older with reversible obstructive airway disease.

Terbutaline sulphate is chemically, (±)-α-[(tertbutylamino) methyl]-3,5-dihydroxybenzyl alcohol sulphate (2:1) (salt). Terbutaline sulphate is commercially available as a tablet (marketed by Impax and Lannett) in the United States. Terbutaline sulphate is indicated for the prevention and reversal of bronchospasm in patients 12 years of age and older with asthma and reversible bronchospasm associated with bronchitis and emphysema.

Bambuterol is chemically, (RS)-5-[2-(tert-butylamino)-1-hydroxyethyl]benzene-1,3-diyl bis(dimethylcarbamate). It is commercially available as Bambec® tablet (marketed by Astrazeneca) 10 mg and 20 mg tablets. It is indicated for the management of asthma, bronchospasm and/or reversible airways obstruction.

There still exists a need for an effective therapeutic treatment for respiratory disorders like asthma and COPD.

### SUMMARY

The present invention relates to a pharmaceutical composition comprising a TRPA1 antagonist and a beta-2 agonist.

The inventors have surprisingly found that a TRPA1 antagonist and a beta-2 agonist act synergistically in the treatment of respiratory disorders and are more effective and provide better therapeutic value than treatment with either active ingredient alone.

The beta-2 agonist, as contemplated herein, includes salbutamol (INN: Albuterol), levosalbutamol, terbutaline, salmeterol, formoterol, metaproterenol, pirbuterol, bambuterol, procaterol, metaproterenol, fenoterol, bitolterol, indacaterol, vilanterol, arformoterol, olodaterol, abediterol, milveterol, bedoradrine, rirodrine clenbuterol, reproterol, PF-610355, GSK-597901, GSK-159802 and GSK-678007 or salts thereof. The salt may be present in the form of their isomers, polymorphs, and solvates, including hydrates, all of which are included in the scope of the invention. Preferably, the beta-2 agonist includes salbutamol, levosalbutamol, terbutaline, salmeterol, formoterol, arformoterol, indacaterol and bambuterol or salts thereof.

In an embodiment, the present invention relates to a pharmaceutical composition comprising synergistically effective amount of a TRPA1 antagonist having an IC₅₀ for inhibiting human TRPA1 receptor activity of less than 1 micromolar, and a beta-2 agonist. Preferably, the TRPA1 antagonist of the present invention has an IC₅₀ for inhibiting human TRPA1 receptor activity of less than 500 nanomolar, or more preferably less than 250 nanomolar, as measured by a method described herein.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

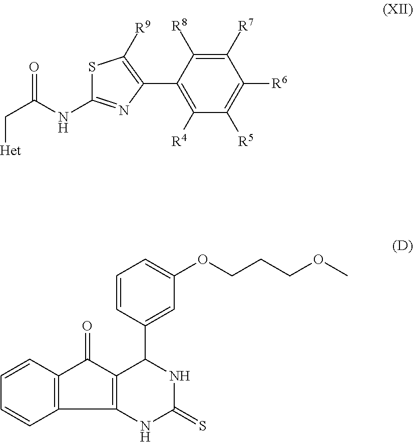

or a pharmaceutically-acceptable salt thereof, wherein, ‘Het’ is selected from the group consisting of

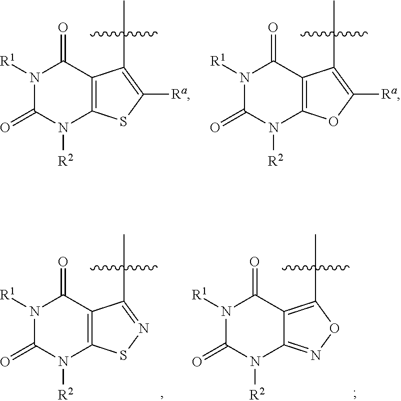

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and a beta-2 agonist.

In yet another embodiment, the present invention relates to a pharmaceutical composition comprising synergistically effective amount of a TRPA1 antagonist having structure of formula:

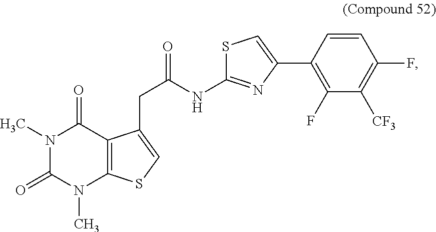 [SMILES: Cn1c(=O)c2c(CC(=O)Nc3nc(-c4ccc(F)c(C(F)(F)F)c4F)cs3)csc2n(C)c1=O]

and a beta-2 agonist.

In another embodiment, there is provided a pharmaceutical composition comprising synergistically effective amount of a TRPA1 antagonist having an IC₅₀ for inhibiting human TRPA1 receptor activity of less than 1 micromolar and a beta-2 agonist in a weight ratio ranging from about 1:0.0001 to about 1:4000.

In an embodiment, the present invention relates to a method of treating a respiratory disorder in a subject, said method comprising administering to the subject the pharmaceutical composition comprising synergistically effective amount of a TRPA1 antagonist having an IC₅₀ for inhibiting human TRPA1 receptor activity of less than 1 micromolar and a beta-2 agonist. In an aspect of this embodiment, the TRPA1 antagonist has an IC₅₀ for inhibiting human TRPA1 receptor activity of less than 1 micromolar having structure of formulae: (XII) or (D)

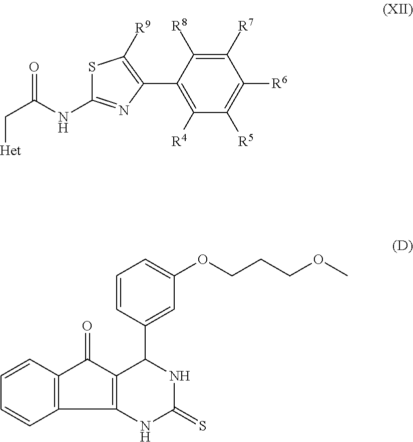

or a pharmaceutically-acceptable salt thereof, wherein, ‘Het’ is selected from the group consisting of

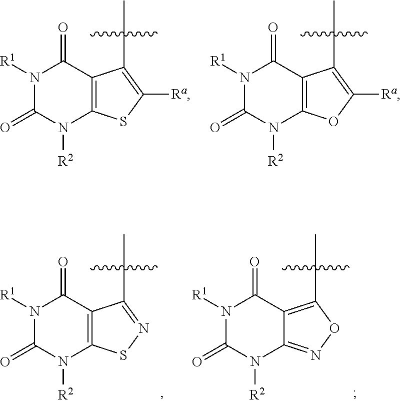

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

The respiratory disorder, in the context of present invention, includes but is not limited to airway inflammation, asthma, emphysema, bronchitis, COPD, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy.

In a further embodiment, the present invention relates to a method of treating a respiratory disorder in a subject, said method comprising administering the subject a pharmaceutical composition comprising synergistically effective amount of a TRPA1 antagonist having an IC₅₀ for inhibiting human TRPA1 receptor activity of less than 1 micromolar and beta-2 agonist selected from a group consisting of salbutamol, levosalbutamol, terbutaline, salmeterol, formoterol, arformoterol, indacaterol and bambuterol or salts thereof.

In a further embodiment, the present invention relates to use of synergistically effective amount of a TRPA1 antagonist having an IC₅₀ for inhibiting human TRPA1 receptor activity of less than 1 micromolar and a beta-2 agonist in the preparation of a pharmaceutical composition of the present invention for the treatment of a respiratory disorder in a subject. In an aspect of this embodiment, the TRPA1 antagonist has an IC₅₀ for inhibiting human TRPA1 receptor activity of less than 1 micromolar having structure of formulae: (XII) or (D)

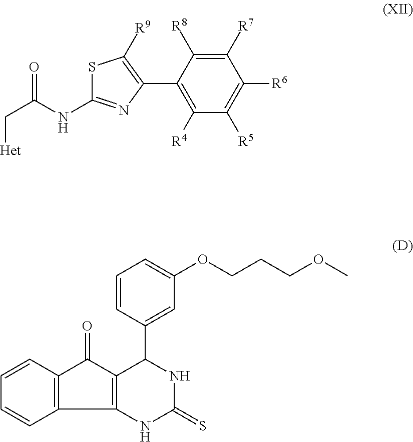

or a pharmaceutically-acceptable salt thereof, wherein, ‘Het’ is selected from the group consisting of

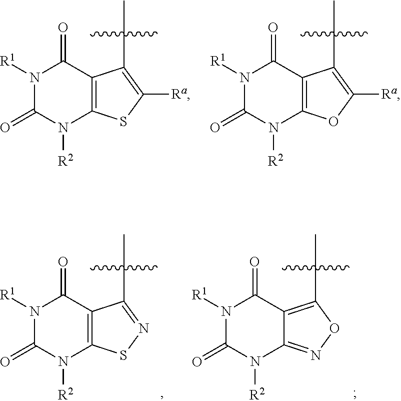

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a pharmaceutical composition comprising synergistically effective amount of a TRPA1 antagonist having an IC₅₀ for inhibiting human TRPA1 receptor activity of less than 1 micromolar and a beta-2 agonist for the treatment of a respiratory disorder in a subject.

In an embodiment, the present invention relates to a pharmaceutical composition comprising synergistically effective amount of a TRPA1 antagonist having structure of formula:

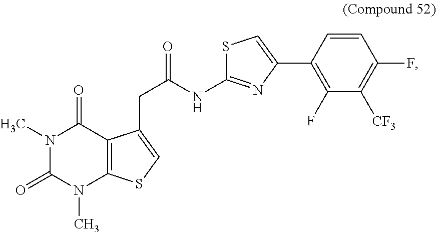 [SMILES: Cn1c(=O)c2c(CC(=O)Nc3nc(-c4ccc(F)c(C(F)(F)F)c4F)cs3)csc2n(C)c1=O]

and a beta-2 agonist selected from a group consisting of salbutamol, levosalbutamol, terbutaline, salmeterol, formoterol, arformoterol, indacaterol and bambuterol or salts thereof. In an aspect of this embodiment, the pharmaceutical composition is a fixed dose combination.

In another aspect of this embodiment, the composition is for oral administration and the TRPA1 antagonist and the beta-2 agonist are present in a weight ratio from about 1:0.01 to about 1:5.

In yet another aspect of this embodiment, the composition is for inhalation administration and the TRPA1 antagonist and the beta-2 agonist are present in a weight ratio from about 1:0.001 to about 1:300.

In an embodiment, the present invention relates to a method of treating a respiratory disorder in a subject, said method comprising administering to the subject the pharmaceutical composition comprising synergistically effective amount of a TRPA1 antagonist having structure of formula:

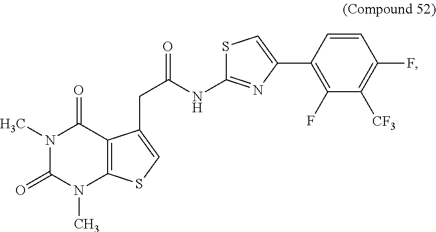 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof.

In an embodiment, the present invention relates to a method of treating a respiratory disorder by improving lung function in a subject, said method comprising administering to the subject the pharmaceutical composition comprising synergistically effective amount of a TRPA1 antagonist having structure of formula:

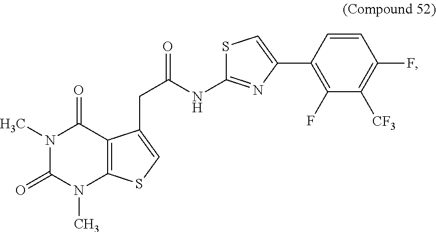 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof. In another aspect of this embodiment, the respiratory disorder is asthma.

In an embodiment, the present invention relates to a method of improving lung function in a subject, said method comprising administering to the subject the pharmaceutical composition comprising synergistically effective amount of a TRPA1 antagonist having structure of formula:

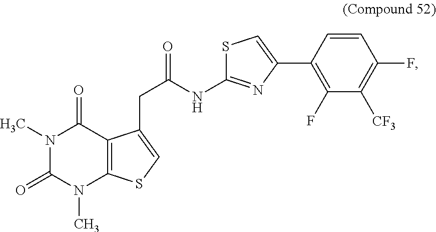 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof.

In another embodiment, the present invention relates to use of synergistically effective amount of a TRPA1 antagonist having structure of formula:

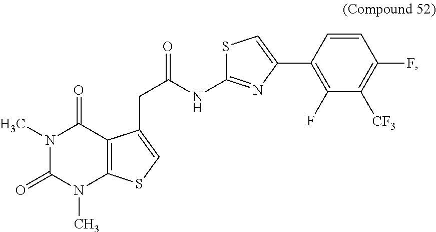 [SMILES: Cn1c(=O)c2c(CC(=O)Nc3nc(-c4ccc(F)c(C(F)(F)F)c4F)cs3)csc2n(C)c1=O]

and beta-2 agonist in the preparation of a pharmaceutical composition of the present invention for the treatment of a respiratory disorder in a subject. In an aspect of this embodiment, the beta-2 agonist selected from a group consisting of salbutamol, levosalbutamol, terbutaline, salmeterol, formoterol, arformoterol, indacaterol and bambuterol or salts thereof.

In a further embodiment, the present invention relates to a pharmaceutical composition comprising synergistically effective amount of a TRPA1 antagonist having structure of formula:

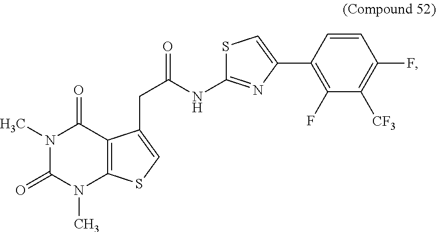 [SMILES: Cn1c(=O)c2c(CC(=O)Nc3nc(-c4ccc(F)c(C(F)(F)F)c4F)cs3)csc2n(C)c1=O]

and a beta-2 agonist for the treatment of a respiratory disorder in a subject.

### BRIEF DESCRIPTION OF THE DRAWINGS

FIG. 1 is a line graph showing the effect of Compound 52 and salmeterol on methacholine induced bronchoconstriction measured as Penh in ovalbumin sensitized female BALB/c mice.

FIG. 2 is a line graph showing the effect of Compound 52 and formoterol on methacholine induced bronchoconstriction measured as Penh in ovalbumin sensitized female BALB/c mice.

FIG. 3 is a line graph showing the effect of Compound 52 and salbutamol on methacholine induced bronchoconstriction measured as Penh in female BALB/c mice.

### DETAILED DESCRIPTION

### Definitions

The terms used herein are defined as follows. If a definition set forth in the present application and a definition set forth later in a non-provisional application claiming priority from the present provisional application are in conflict, the definition in the non-provisional application shall control the meaning of the terms.

The term “effective amount” or “therapeutically effective amount” denotes an amount of an active ingredient that, when administered to a subject for treating a respiratory disorder, produces an intended therapeutic benefit in a subject. The effective amount of TRPA1 antagonist as described herein ranges from about 0.1 mcg/kg to about 20 mg/kg, and preferably from about 1 mcg/kg to about 15 mg/kg. The therapeutically effective amount of salmeterol or its salt ranges from about 10 mcg to about 1 mg, and preferably from about 20 mcg to about 500 mcg, and more preferably from about 50 mcg to about 100 mcg. The therapeutically effective amount of formoterol or its salt ranges from about 1 mcg to about 100 mcg, and preferably from about 5 mcg to about 50 mcg, and more preferably from about 10 mcg to about 25 mcg. The therapeutically effective amount of indacaterol or its salt ranges from about 50 mcg to about 500 mcg; and preferably from about 100 mcg to about 400 mcg, and more preferably from about 150 mcg to about 300 mcg. The therapeutically effective amount of salbutamol or its salt ranges from about 0.01 mg to about 30 mg, and preferably from about 0.05 mg to about 20 mg. The therapeutically effective amount of levosalbutamol or its salt ranges from about 0.1 mg to about 5 mg, and preferably from about 0.2 mg to about 4.5 mg, and more preferably from about 0.3 mg to about 4 mg. The therapeutically effective amount of terbutaline or its salt ranges from about 0.1 mg to about 1 mg, and preferably from about 0.2 mg to about 0.75 mg, and more preferably from about 0.25 mg to about 0.5 mg. The therapeutically effective ranges of actives are given as above, although larger or smaller amount are not excluded if they fall within the scope of the definition of this paragraph.

The term “active ingredient” (used interchangeably with “active” or “active substance” or “drug”) as used herein includes a TRPA1 antagonist, a beta-2 agonist or a pharmaceutically acceptable salt thereof. Preferably, the active ingredient includes TRPA1 antagonist having a human IC₅₀ value of less than 1 micromolar, salmeterol, formoterol, arformoterol, indacaterol, salbutamol, levosalbutamol, bambuterol or terbutaline or its salt.

The IC₅₀ value is believed to be measure of the effectiveness of a compound in inhibiting biological or biochemical function. This quantitative measure generally indicates molar concentration of a particular compound (or substance) is needed to inhibit a given biological process by half. In other words, it is the half maximal (50%) inhibitory concentration (IC) of the compound. The IC₅₀ of a drug compound (or active substance) can be determined by constructing a concentration-response curve so as to examine the effect of different concentrations of antagonist on reversing agonist activity. IC₅₀ values can be calculated for a given antagonist by determining the concentration needed to inhibit half of the maximum biological response of the agonist. IC₅₀ values can be used to compare the potency of two antagonists.

By “salt” or “pharmaceutically acceptable salt”, it is meant those salts and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include the sodium, calcium, potassium and magnesium salts.

The term “treating” or “treatment” as used herein also covers the prophylaxis, mitigation, prevention, amelioration, or suppression of a disorder modulated by the TRPA1 receptor, or the beta-2 adrenergic receptor, or by a combination of the two in a subject.

The respiratory disorder includes but is not limited to airway inflammation, asthma, emphysema, bronchitis, COPD, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy. Preferably, the respiratory disorder is asthma or COPD.

In the context of present invention, the term “improving lung function” or “improvement in lung function” refers to enhancing or improving the declined lung function in a subject having a respiratory disorder by one or more of the following mechanisms, but not limited to,—inhibiting bronchoconstriction, preventing bronchoconstriction, inducing bronchodilation, reducing airway hyper-reactivity/responsiveness by suppression of airway inflammation or reducing exacerbations—in said subject.

Lung function generally means how well one's lungs work. Various tests are used to assess the lung function in human. For example, spirometry, which is the most commonly used lung function test, measures specifically the amount (volume) and/or speed (flow) of air that can be inhaled and exhaled. Typically, spirometric measurements involve determination on certain functional parameters such as forced expiratory volume (FEV), forced vital capacity (FVC), forced expiratory flow, peak expiratory flow, and the like.

Asthma in humans typically manifests as bronchoconstrictive responses namely early allergen response (EAR) and late asthmatic response (LAR). EAR occurs 15-30 minutes post allergen exposure whereas LAR occurs after 3-5 hours, reaches maximum at 6-12 hours and may persist up to 24 hours. (Clin. Allergy. 1977, 7:503-513; Clin. Exp. Allergy. 1991, 21:3-7.) These bronchoconstrictive responses are believed to result in declined lung/pulmonary function. Such lung function decline can also be simulated in rodent models of allergic asthma and is measured as Penh (enhanced pause). These animal models are characterised by inflammatory infiltration and a biphasic bronchoconstrictor response (EAR and LAR) (Thorax 2012; 67:19-25). In conscious animals, an EAR is followed by LAR and both can be subjectively evidenced by audible (wheeze) and visual signs of respiratory distress associated with quantifiable changes in lung function that can be measured non-invasively as Penh (Am. J. Respir. Crit. Care Med. 2005; 172: 962-71). Thus increased Penh is an indicator of decreased pulmonary/lung function during EAR and LAR and is a close correlate of lung resistance (Am. J. Respir. Crit. Care Med. 1997, 156:766-775). Generally, if the Penh is significantly reduced (p<0.05 or less) in the drug treated animals as compared to the vehicle treated animals, then the observed effect is considered as significant improvement in the lung function in said animals.

The term “subject” includes mammals like human and other animals, such as domestic animals (e.g., household pets including cats and dogs) and non-domestic animals (such as wildlife). Preferably, the subject is a human.

By “pharmaceutically acceptable excipients”, it is meant any of the components of a pharmaceutical composition other than the actives and which are approved by regulatory authorities or are generally regarded as safe for human or animal use.

The term “synergistic” or “synergy”, as used herein, refers to a combination exhibiting an effect greater than would be expected from the sum of the effects of the individual components of the combination alone. The term “synergistic” or “synergy” with regard to the combination of a TRPA1 antagonist with a leukotriene receptor antagonist which is used in the treatment of a respiratory disorder (for example, in the form of a pharmaceutical composition, a combination product or a kit according to the invention) refers to an efficacy for the treatment of the respiratory disorder that is greater than would be expected from the sum of their individuals effects. The advantages for the synergistic combinations of the present invention include, but are not limited to, lowering the required dose of one or more of the active compounds of the combination, reducing the side effects of one or more of the active compounds of the combination and/or rendering one or more of the active compounds more tolerable to the subject in need of treatment of the respiratory disorder.

### Combinations

The present invention relates to a pharmaceutical composition comprising a TRPA1 antagonist and a beta-2 agonist.

The inventors have surprisingly found that a TRPA1 antagonist and a beta-2 agonist act synergistically in the treatment of respiratory disorders, and are more effective and provide better therapeutic value than treatment with either active ingredient alone.

In an aspect, TRPA1 antagonists useful in the context of the invention, are selected from one of the following formulae: (A) or (B) or (C) or (D)

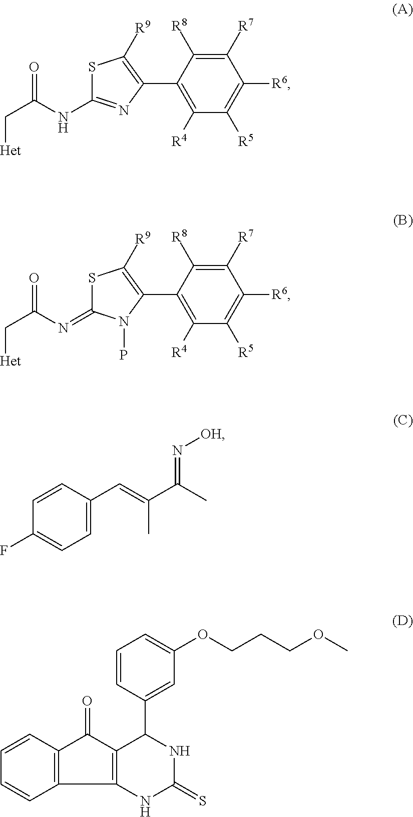

or a pharmaceutically-acceptable salt thereof, wherein, ‘Het’ is selected from the group consisting of

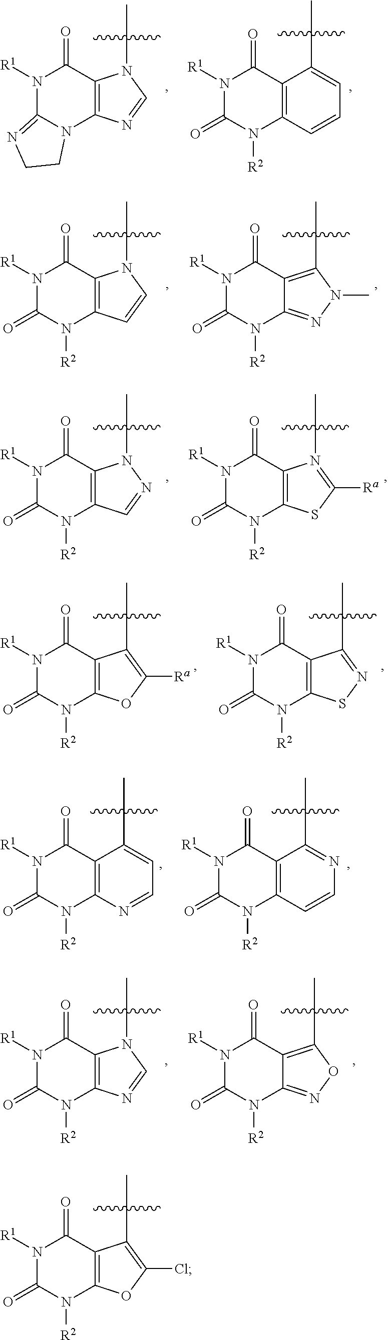

P is selected from

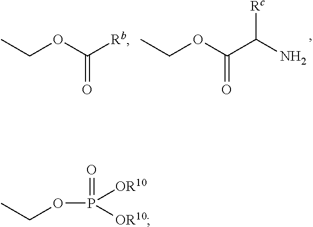

R¹, R² and R^{a}, which may be the same or different, are each independently hydrogen or (C₁-C₄) alkyl;

R^{b} and R^{c} independently selected from hydrogen, substituted or unsubstituted alkyl arylalkyl, amino acid and heterocyclic ring;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R¹⁰ is selected from hydrogen, alkyl, arylalkyl and pharmaceutically acceptable cation.

In one aspect, TRPA1 antagonists useful in the context of the invention are selected from those compounds generically or specifically disclosed in WO2009144548. Accordingly, a TRPA1 antagonist useful in the context of the invention has the formula (I):

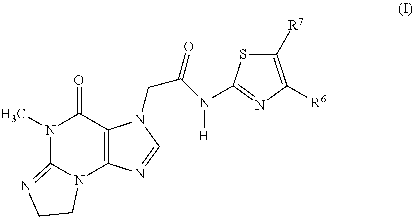

or a pharmaceutically acceptable salt thereof,
wherein,

R⁶ represents hydrogen, substituted or unsubstituted alkyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R⁷ independently represents hydrogen or alkyl.

Few representative TRPA1 antagonists useful in the methods of the invention are mentioned below:

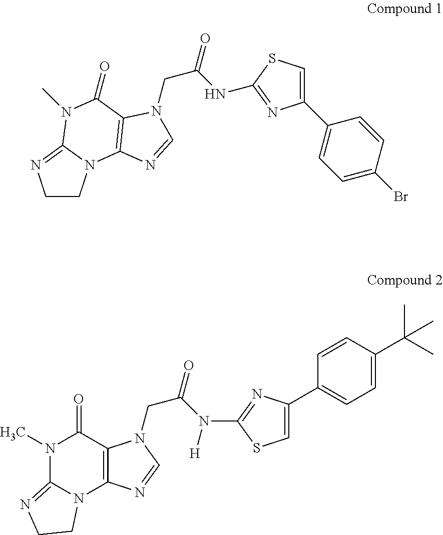 [SMILES: CN1C(=O)c2c(ncn2CC(=O)Nc2nc(-c3ccc(Br)cc3)cs2)N2CCN=C12 ; CN1C(=O)c2c(ncn2CC(=O)Nc2nc(-c3ccc(C(C)(C)C)cc3)cs2)N2CCN=C12]

The preparation of above said compounds is described in WO2009144548.

In another aspect, TRPA1 antagonists useful in the context of the invention are selected from those compounds generically or specifically disclosed in WO2010004390. Accordingly, TRPA1 antagonist useful in the context of the invention has the formula (II):

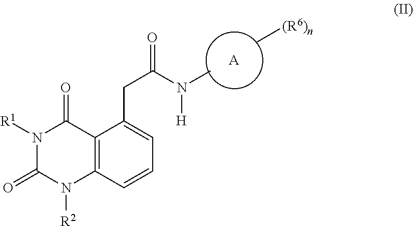

or pharmaceutically acceptable salts thereof,
wherein,

at each occurrence R¹ and R² is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, (CR^{x}R^{y})ₙOR^{x}, COR^{x}, COOR^{x}, CONR^{x}R^{y}, SO₂NR^{x}R^{y}, NR^{x}R^{y}, NR^{x}(CR^{x}R^{y})ₙOR^{x}, NR^{x}(CR^{x}R^{y})ₙCN(CH₂)ₙNR^{x}R^{y}, (CH₂)ₙCHR^{x}R^{y}, (CR^{x}R^{y})NR^{x}R^{y}, NR^{x}(CR^{x}R^{y})ₙCONR^{x}R^{y}, (CH₂)ₙNHCOR^{x} and (CH₂)ₙNH(CH₂)ₙSO₂R^{x}, (CH₂)ₙNHSO₂R^{x};

R^{x} and R^{y} are independently selected from hydrogen, hydroxyl, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R^{x} and R^{y} may be joined together to form an optionally substituted 3 to 7 membered saturated, unsaturated or partially saturated cyclic ring, which may optionally include at least two heteroatoms selected from O, NR^{a} or S;

ring A is selected from phenyl, pyridinyl, pyrazolyl, thiazolyl and thiadiazolyl;

each occurrence of R⁶ is independently hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl,

R^{x} and R^{y} are independently selected from hydrogen, hydroxyl, halogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, and substituted or unsubstituted heteroarylalkyl;

at each occurrence of ‘n’ is independently selected from 1 to 5.

According to one aspect, specifically provided are compounds of the formula (Ha)

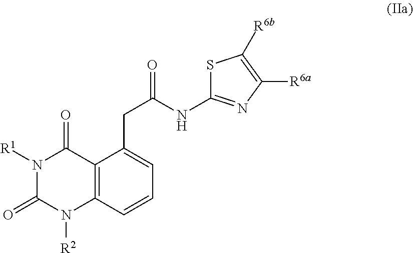

or pharmaceutically acceptable salts thereof,
wherein,

R¹ and R² are as defined above for the compound of formula (II);

R^{6a} and R^{6b} are independently selected from hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl, —C(O)OR^{x}, —OR^{x}, —C(O)NR^{x}R^{y}, —C(O)R^{x}, —SO₂R^{x}, —SO₂—NR^{x}R^{y}.

Few representative TRPA1 antagonists useful in the context of the invention are mentioned below:

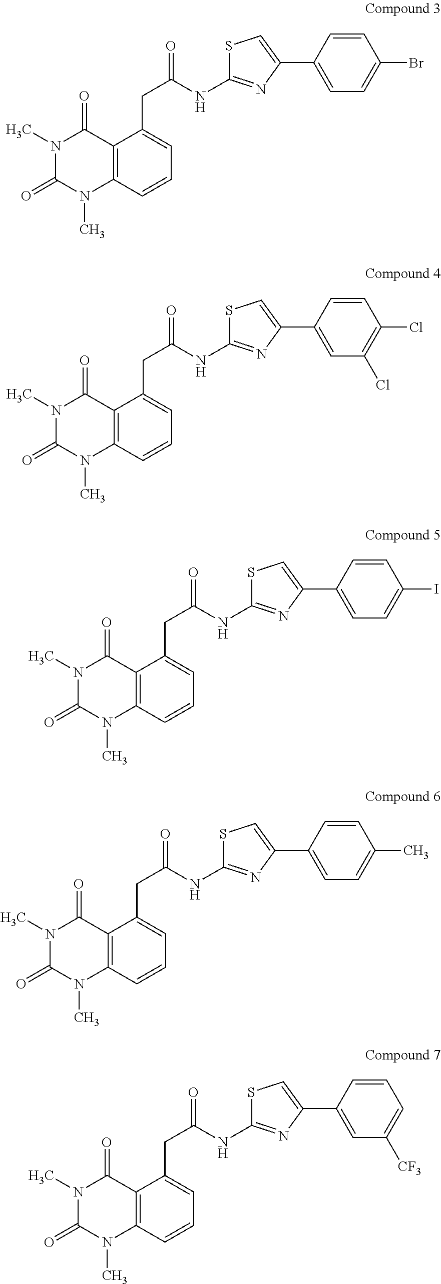

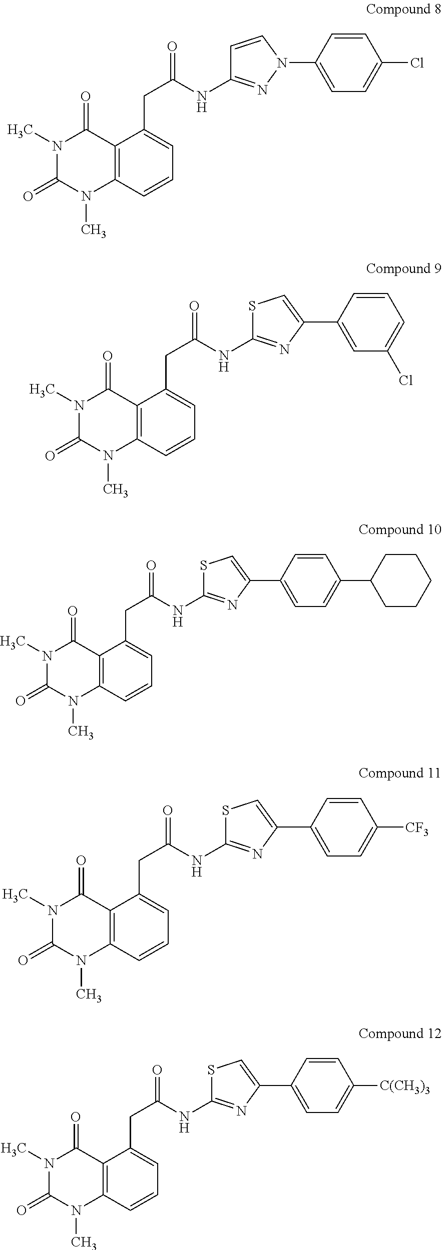 [SMILES: Cn1c(=O)c2c(CC(=O)Nc3ccn(-c4ccc(Cl)cc4)n3)cccc2n(C)c1=O ; Cn1c(=O)c2c(CC(=O)Nc3nc(-c4cccc(Cl)c4)cs3)cccc2n(C)c1=O ; Cn1c(=O)c2c(CC(=O)Nc3nc(-c4ccc(C5CCCCC5)cc4)cs3)cccc2n(C)c1=O ; Cn1c(=O)c2c(CC(=O)Nc3nc(-c4ccc(C(F)(F)F)cc4)cs3)cccc2n(C)c1=O ; Cn1c(=O)c2c(CC(=O)Nc3nc(-c4ccc(C(C)(C)C)cc4)cs3)cccc2n(C)c1=O]

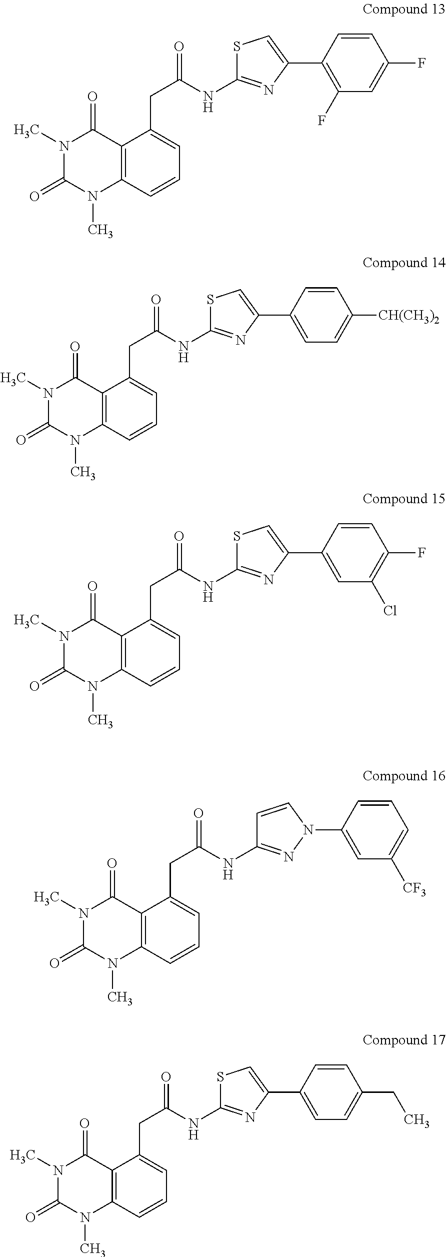 [SMILES: Cn1c(=O)c2c(CC(=O)Nc3nc(-c4ccc(F)cc4F)cs3)cccc2n(C)c1=O ; CC(C)c1ccc(-c2csc(NC(=O)Cc3cccc4c3c(=O)n(C)c(=O)n4C)n2)cc1 ; Cn1c(=O)c2c(CC(=O)Nc3nc(-c4ccc(F)c(Cl)c4)cs3)cccc2n(C)c1=O ; Cn1c(=O)c2c(CC(=O)Nc3ccn(-c4cccc(C(F)(F)F)c4)n3)cccc2n(C)c1=O ; CCc1ccc(-c2csc(NC(=O)Cc3cccc4c3c(=O)n(C)c(=O)n4C)n2)cc1]

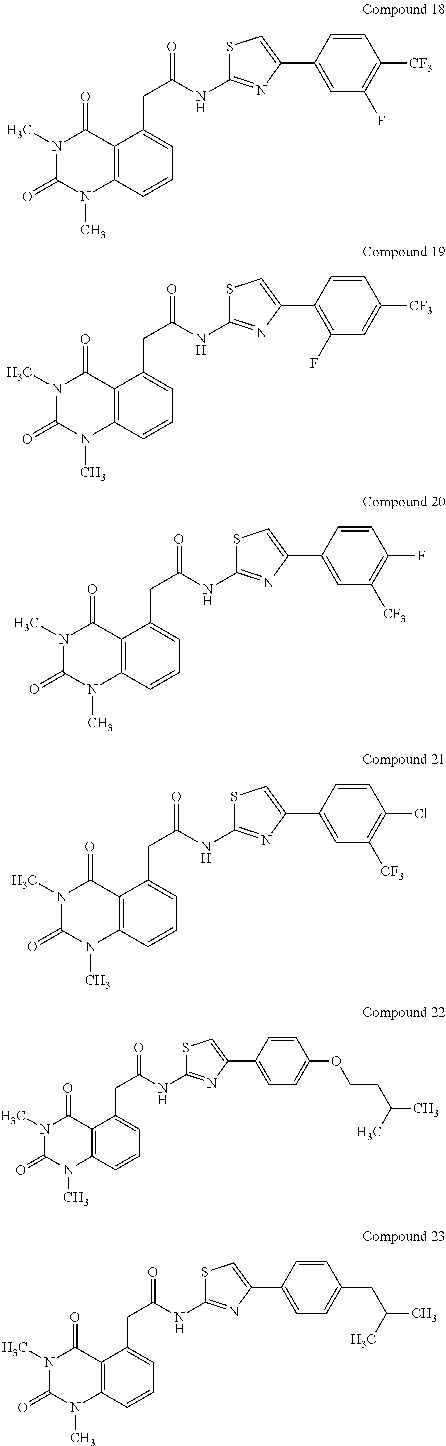

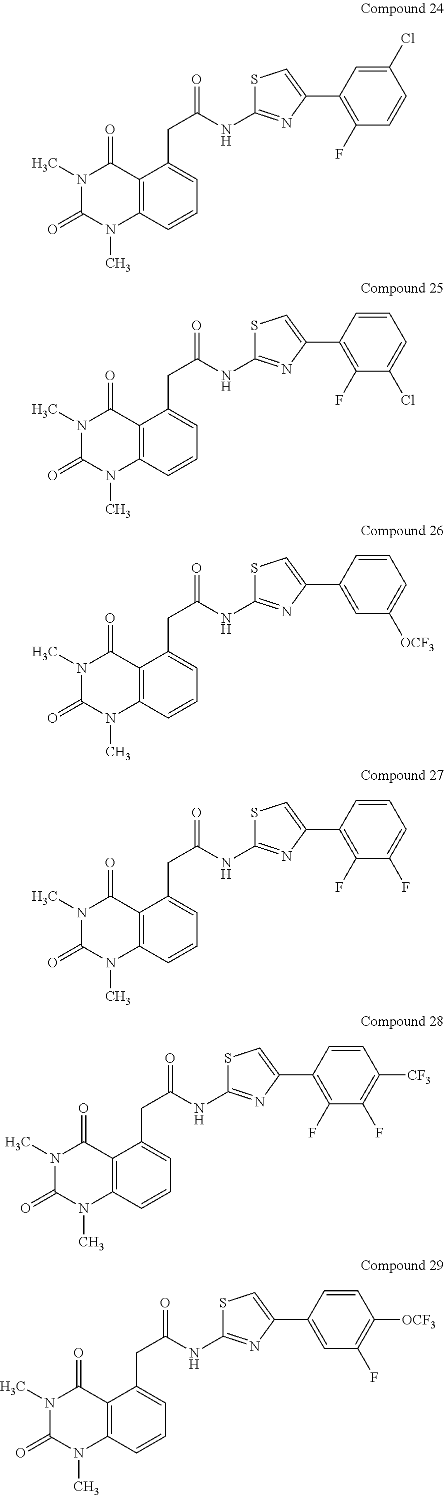 [SMILES: Cn1c(=O)c2c(CC(=O)Nc3nc(-c4cc(Cl)ccc4F)cs3)cccc2n(C)c1=O ; Cn1c(=O)c2c(CC(=O)Nc3nc(-c4cccc(Cl)c4F)cs3)cccc2n(C)c1=O ; Cn1c(=O)c2c(CC(=O)Nc3nc(-c4cccc(OC(F)(F)F)c4)cs3)cccc2n(C)c1=O ; Cn1c(=O)c2c(CC(=O)Nc3nc(-c4cccc(F)c4F)cs3)cccc2n(C)c1=O ; Cn1c(=O)c2c(CC(=O)Nc3nc(-c4ccc(C(F)(F)F)c(F)c4F)cs3)cccc2n(C)c1=O ; Cn1c(=O)c2c(CC(=O)Nc3nc(-c4ccc(OC(F)(F)F)c(F)c4)cs3)cccc2n(C)c1=O]

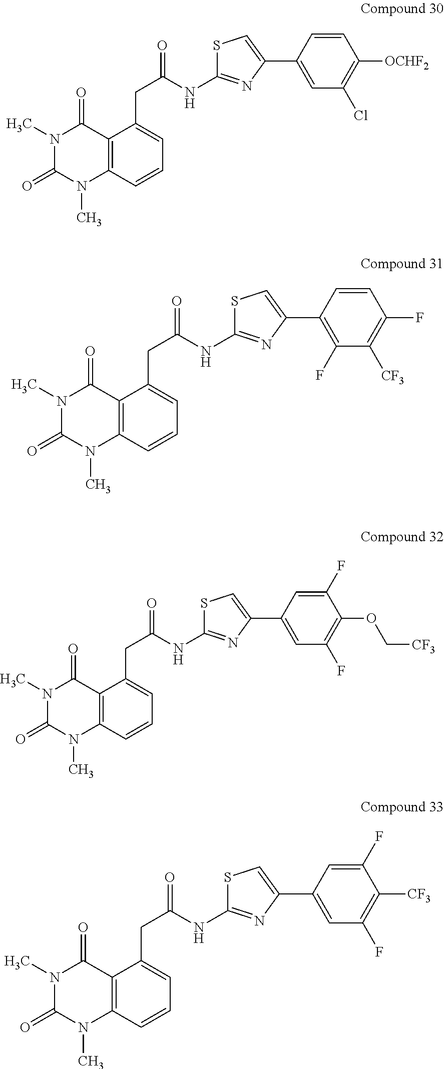 [SMILES: Cn1c(=O)c2c(CC(=O)Nc3nc(-c4ccc(OC(F)F)c(Cl)c4)cs3)cccc2n(C)c1=O ; Cn1c(=O)c2c(CC(=O)Nc3nc(-c4ccc(F)c(C(F)(F)F)c4F)cs3)cccc2n(C)c1=O ; Cn1c(=O)c2c(CC(=O)Nc3nc(-c4cc(F)c(OCC(F)(F)F)c(F)c4)cs3)cccc2n(C)c1=O ; Cn1c(=O)c2c(CC(=O)Nc3nc(-c4cc(F)c(C(F)(F)F)c(F)c4)cs3)cccc2n(C)c1=O]

The preparation of above said compounds is described in WO2010004390.

In one aspect, TRPA1 antagonists useful in the context of the invention are selected from those compounds generically or specifically disclosed in WO2010109287. Accordingly, TRPA1 antagonist useful in the context of the invention has the formula (III):

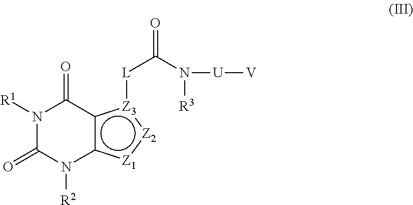

or a pharmaceutically acceptable salt thereof,
wherein,

Z₁ is NR^{a} or CR^{a};

Z₂ is NR^{b} or CR^{b};

Z₃ is N or C;

with the proviso that when Z₂ is CR^{b} then both Z₁ and Z₃ are not nitrogen at the same time;

at each occurrence, R^{a} and R^{b} which may be same or different, are independently selected from hydrogen, hydroxyl, cyano, halogen, substituted or unsubstituted alkyl, haloalkyl, alkenyl, alkynyl, cycloalkyl, cycloalkylalkyl, —(CR^{x}R^{y})ₙOR^{x}, —COR^{x}, —COOR^{x}, —CONR^{x}R^{y}, —S(O)ₘNR^{x}R^{y}, —NR^{x}R^{y}, —NR^{x}(CR^{x}R^{y})ₙOR^{x}, —(CH₂)ₙNR^{x}R^{y}, —(CH₂)ₙCHR^{x}R^{y}, —(CH₂)NR^{x}R^{y}, —NR^{x}(CR^{x}R^{y})ₙCONR^{x}R^{y}, —(CH₂)ₙNHCOR^{x}, —(CH₂)ₙNH(CH₂)ₙSO₂R^{x} and (CH₂)ₙNHSO₂R^{x};

alternatively either of R^{a} or R^{b} is absent;

R¹ and R², which may be same or different, are independently selected from hydrogen, hydroxyl, substituted or unsubstituted alkyl, haloalkyl, alkenyl, alkynyl, cycloalkyl, cycloalkylalkyl, arylalkyl, (CR^{x}R^{y})ₙOR^{x}, COR^{x}, COOR^{x}, CONR^{x}R^{y}, (CH₂)ₙNR^{x}R^{y}, (CH₂)ₙCHR^{x}R^{y}, (CH₂)NR^{x}R^{y} and (CH₂)ₙNHCOR^{x};

R³ is selected from hydrogen, substituted or unsubstituted alkyl, alkenyl, haloalkyl, alkynyl, cycloalkyl, cycloalkylalkyl, cycloalkenyl;

L is a linker selected from —(CR^{x}R^{y})ₙ—, —O—(CR^{x}R^{y})ₙ—, —C(O)—, —NR^{x}—, —S(O)ₘNR^{x}—, —NR^{x}(CR^{x}R^{y})ₙ— and —S(O)ₘNR^{x}(CR^{x}R^{y})ₙ;

U is selected from substituted or unsubstituted aryl, substituted or unsubstituted five membered heterocycles selected from the group consisting of thiazole, isothiazole, oxazole, isoxazole, thiadiazole, oxadiazole, pyrazole, imidazole, furan, thiophene, pyroles, 1,2,3-triazoles and 1,2,4-triazole; and substituted or unsubstituted six membered heterocycles selected from the group consisting of pyrimidine, pyridine and pyridazine;

V is selected from hydrogen, cyano, nitro, —NR^{x}R^{y}, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl, —C(O)OR^{x}, —OR^{x}, —C(O)NR^{x}R^{y}, —C(O)R^{x} and —SO₂NR^{x}R^{y}; or U and V together may form an optionally substituted 3 to 7 membered saturated or unsaturated cyclic ring, that may optionally include one or more heteroatoms selected from O, S and N;

at each occurrence, R^{x} and R^{y} are independently selected from the group consisting of hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl; and

at each occurrence ‘m’ and ‘n’ are independently selected from 0 to 2, both inclusive.

Few representative TRPA1 antagonists useful in the context of the invention are mentioned below:

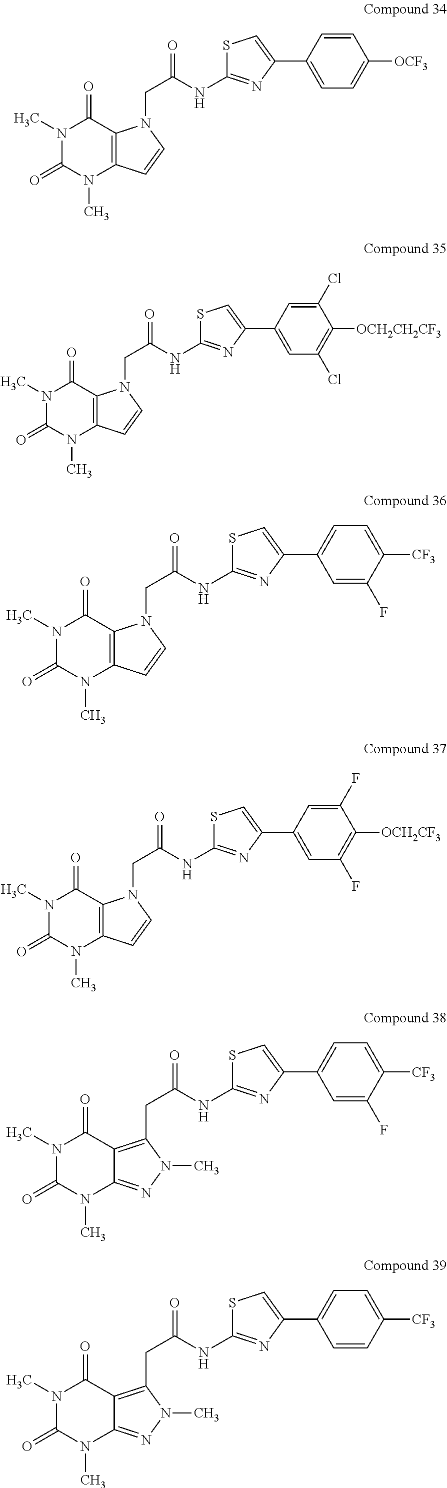

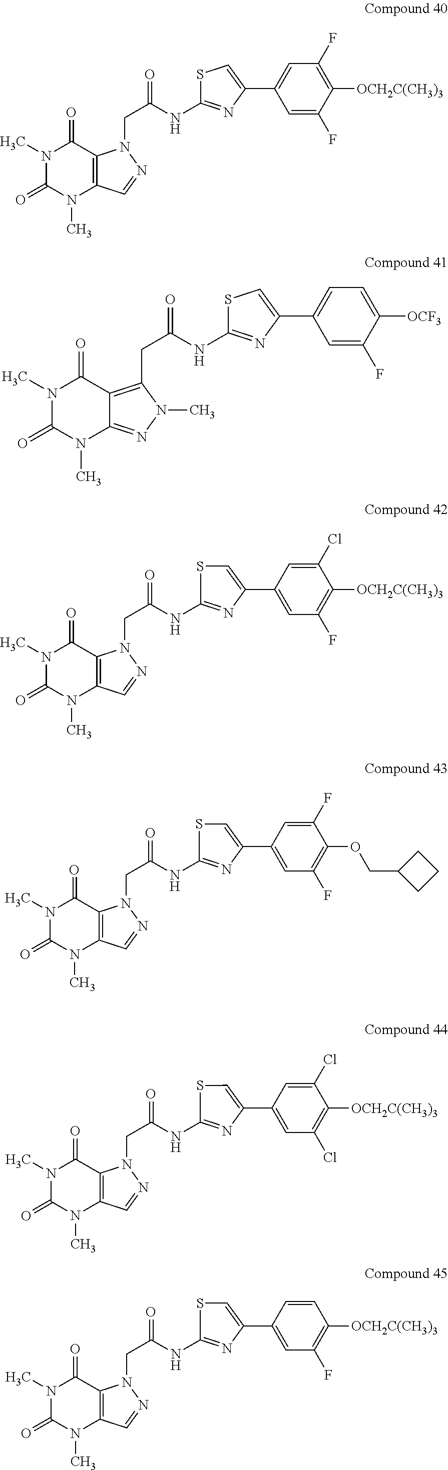

The preparation of above said compounds is described in WO2010109287.

In one aspect, TRPA1 antagonists useful in the context of the invention are selected from those compounds generically or specifically disclosed in WO 2010109334. Accordingly, TRPA1 antagonists useful in the context of the invention has the formula (IV)

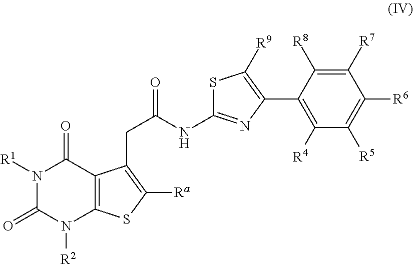

or a pharmaceutically-acceptable salt thereof.

wherein, R¹, R² and R^{a}, which may be the same or different, are each independently hydrogen or (C₁-C₄)alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

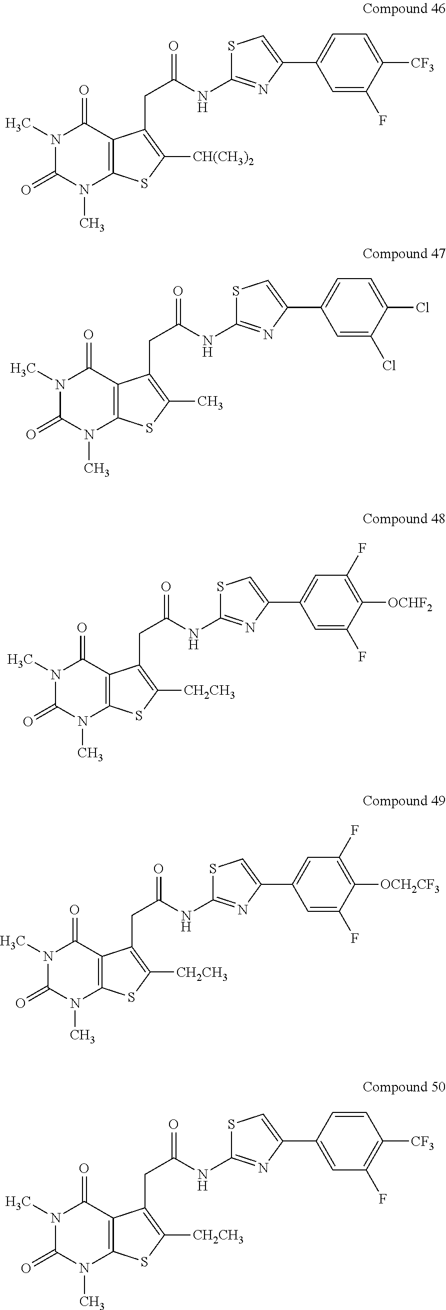

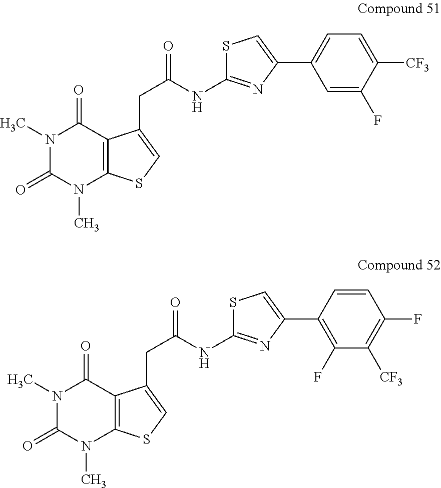 [SMILES: Cn1c(=O)c2c(CC(=O)Nc3nc(-c4ccc(C(F)(F)F)c(F)c4)cs3)csc2n(C)c1=O ; Cn1c(=O)c2c(CC(=O)Nc3nc(-c4ccc(F)c(C(F)(F)F)c4F)cs3)csc2n(C)c1=O]

The preparation of above said compounds is described in WO2010109334.

In one aspect, TRPA1 antagonists useful in the context of the invention are selected from those compounds generically or specifically disclosed in WO2010109329. Accordingly, TRPA1 antagonists useful in the context of the invention has the formula (V)

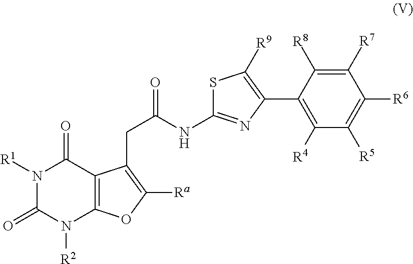

or a pharmaceutically acceptable salt thereof,
wherein, R¹, R² and R^{a} which may be the same or different, are each independently hydrogen or (C₁-C₄) alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

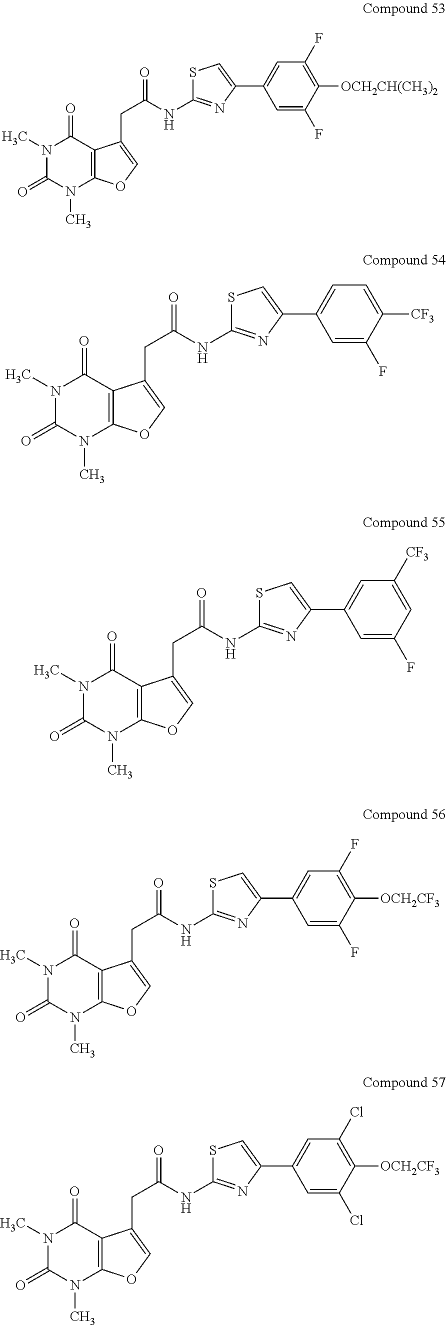

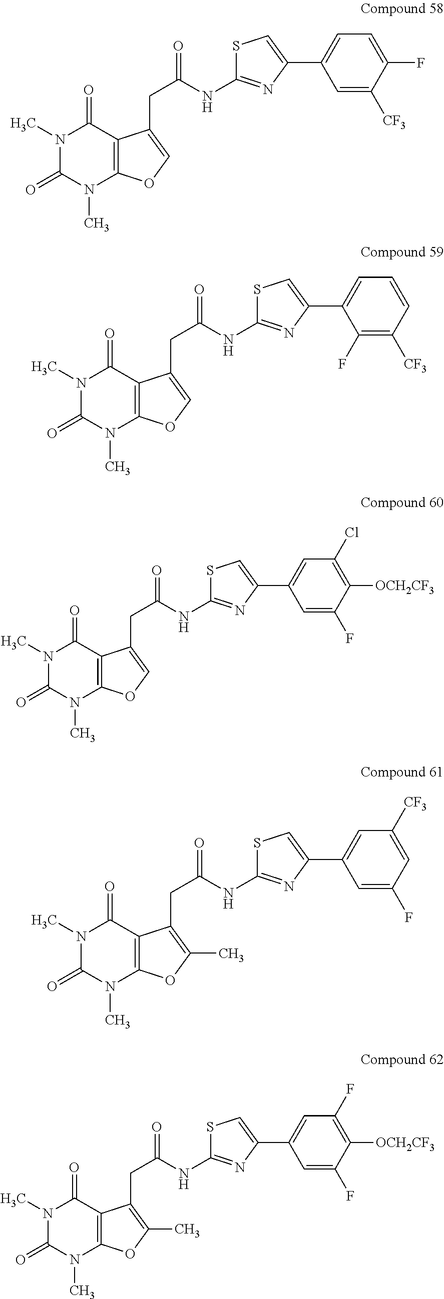

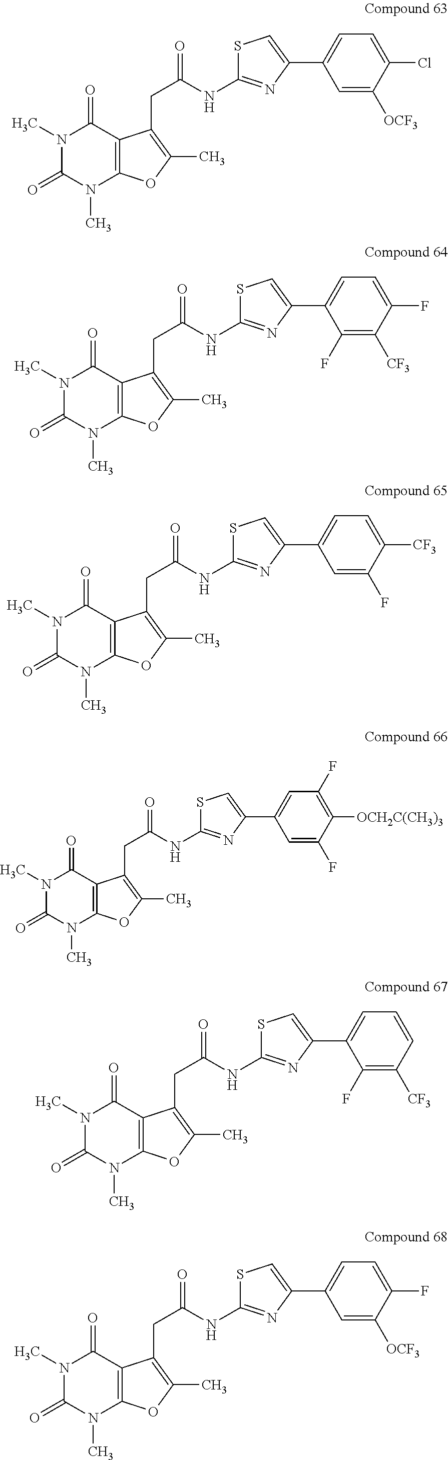

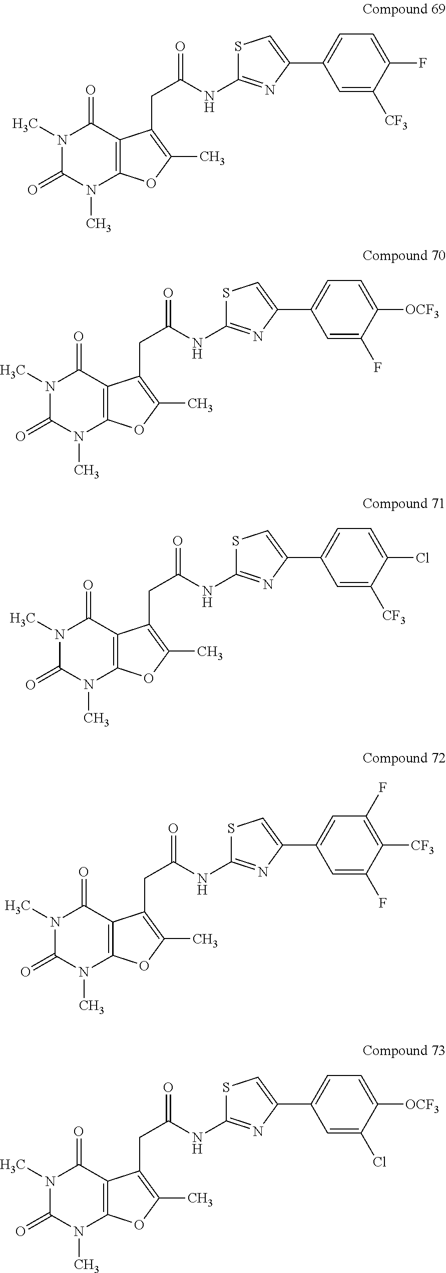 [SMILES: Cc1oc2c(c1CC(=O)Nc1nc(-c3ccc(F)c(C(F)(F)F)c3)cs1)c(=O)n(C)c(=O)n2C ; Cc1oc2c(c1CC(=O)Nc1nc(-c3ccc(OC(F)(F)F)c(F)c3)cs1)c(=O)n(C)c(=O)n2C ; Cc1oc2c(c1CC(=O)Nc1nc(-c3ccc(Cl)c(C(F)(F)F)c3)cs1)c(=O)n(C)c(=O)n2C ; Cc1oc2c(c1CC(=O)Nc1nc(-c3cc(F)c(C(F)(F)F)c(F)c3)cs1)c(=O)n(C)c(=O)n2C ; Cc1oc2c(c1CC(=O)Nc1nc(-c3ccc(OC(F)(F)F)c(Cl)c3)cs1)c(=O)n(C)c(=O)n2C]

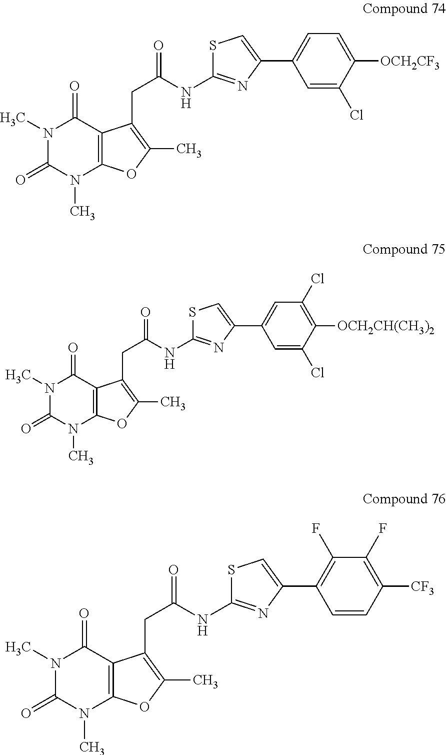

The preparation of above said compounds is described in WO2010109329.

In one aspect, TRPA1 antagonists useful in the context of the invention are selected from those compounds generically or specifically disclosed in WO2010109328. Accordingly, TRPA1 antagonists useful in the context of the invention has the formula (VI)

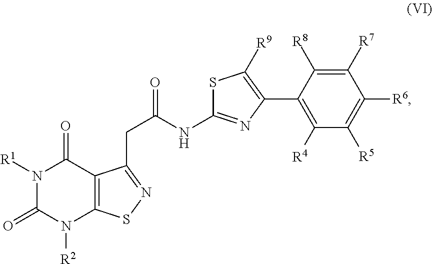

or a pharmaceutically-acceptable salt thereof.
wherein, R¹ and R², which may be the same or different, are each independently hydrogen or (C₁-C₄)alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

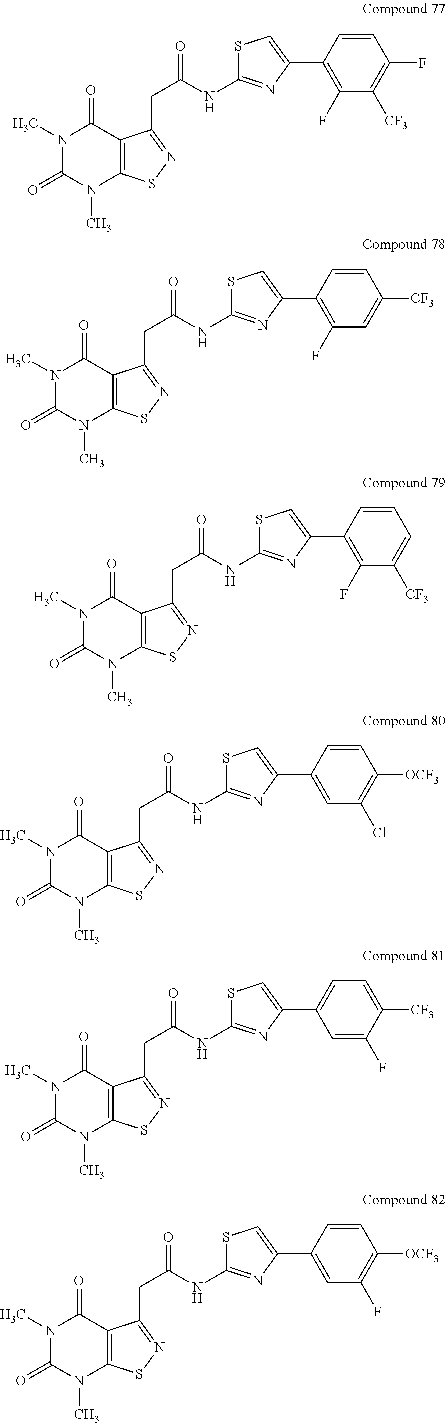 [SMILES: Cn1c(=O)c2c(CC(=O)Nc3nc(-c4ccc(F)c(C(F)(F)F)c4F)cs3)nsc2n(C)c1=O ; Cn1c(=O)c2c(CC(=O)Nc3nc(-c4ccc(C(F)(F)F)cc4F)cs3)nsc2n(C)c1=O ; Cn1c(=O)c2c(CC(=O)Nc3nc(-c4cccc(C(F)(F)F)c4F)cs3)nsc2n(C)c1=O ; Cn1c(=O)c2c(CC(=O)Nc3nc(-c4ccc(OC(F)(F)F)c(Cl)c4)cs3)nsc2n(C)c1=O ; Cn1c(=O)c2c(CC(=O)Nc3nc(-c4ccc(C(F)(F)F)c(F)c4)cs3)nsc2n(C)c1=O ; Cn1c(=O)c2c(CC(=O)Nc3nc(-c4ccc(OC(F)(F)F)c(F)c4)cs3)nsc2n(C)c1=O]

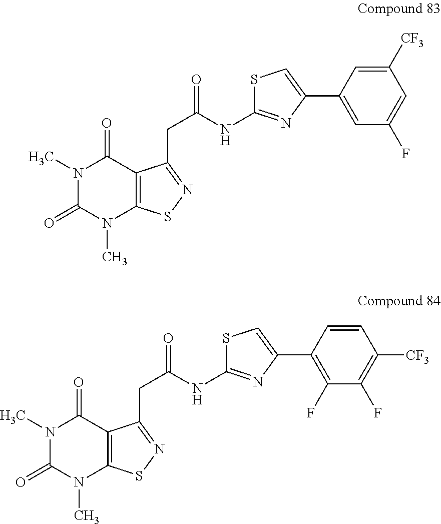 [SMILES: Cn1c(=O)c2c(CC(=O)Nc3nc(-c4cc(F)cc(C(F)(F)F)c4)cs3)nsc2n(C)c1=O ; Cn1c(=O)c2c(CC(=O)Nc3nc(-c4ccc(C(F)(F)F)c(F)c4F)cs3)nsc2n(C)c1=O]

The preparation of above said compounds is described in WO2010109328.

In one aspect, TRPA1 antagonists useful in the context of the invention are selected from those compounds generically or specifically disclosed in WO2010125469. Accordingly, TRPA1 antagonists useful in the context of the invention have the formulas (VIIa, VIIb and VIIc):

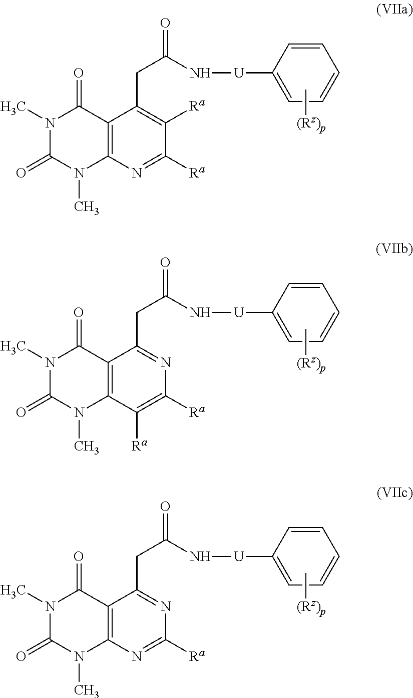

or pharmaceutically acceptable salt thereof,
wherein,

at each occurrence, R^{a} is selected from hydrogen, cyano, halogen, substituted or unsubstituted alkyl, haloalkyl, alkenyl, alkynyl, alkoxy, cycloalkyl and cycloalkylalkyl;

U is substituted or unsubstituted five membered heterocycle, for example selected from the group consisting of

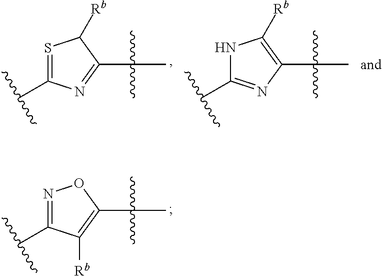

at each occurrence, R^{b} is independently selected from hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

at each occurrence, R^{z} is independently selected from halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring, heterocyclylalkyl, COOR^{x}, CONR^{x}R^{y}, S(O)ₘNR^{x}R^{y}, NR^{x}(CR^{x}R^{y})ₙOR^{x}, (CH₂)ₙNR^{x}R^{y}, NR^{x}(CR^{x}R^{y})ₙCONR^{x}R^{y}, (CH₂)ₙNHCOR^{x}, (CH₂)ₙNH(CH₂)ₙSO₂R^{x} and (CH₂)ₙNHSO₂R^{x};

at each occurrence, R^{x} and R^{y} are independently selected from hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl;

at each occurrence, ‘m’ and ‘n’ are independently selected from 0 to 2, both inclusive; and ‘p’ is independently selected from 0 to 5, both inclusive.

Few representative TRPA1 antagonists useful in the context of the invention are mentioned below:

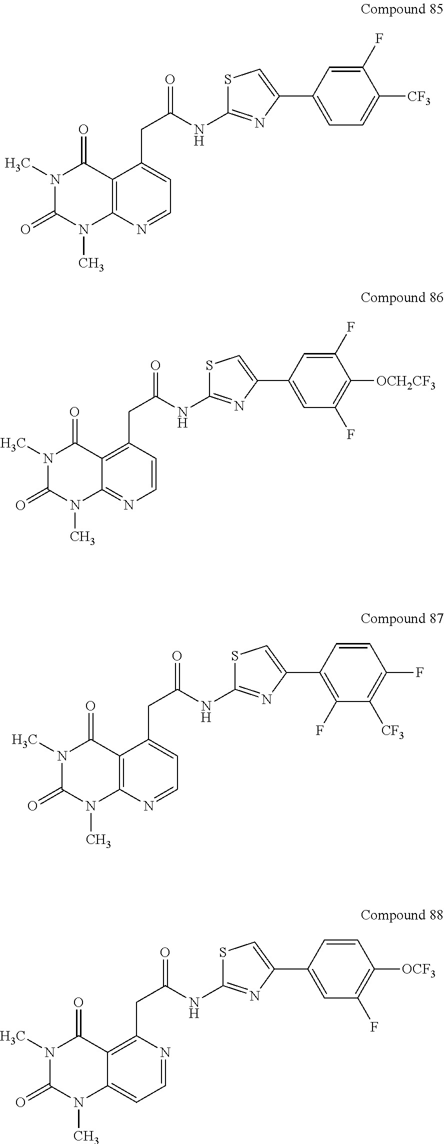

The preparation of above said compounds is described in WO2010125469.

In one aspect, the TRPA1 antagonist useful in the context of the invention is Compound 89:

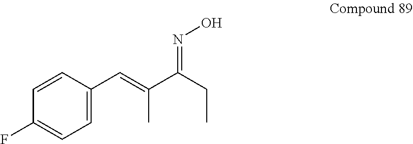

In one embodiment, the TRPA1 antagonist useful in the context of the invention is Compound 90:

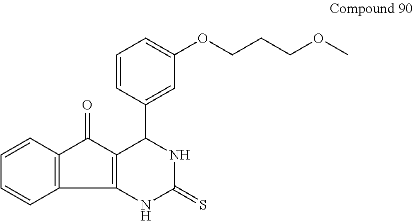 [SMILES: COCCCOc1cccc(C2NC(=S)NC3=C2C(=O)c2ccccc23)c1]

In an embodiment, TRPA1 antagonists useful in the context of the invention has the formula

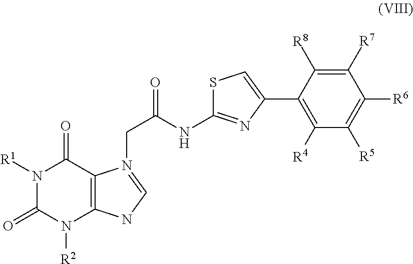

or a pharmaceutically-acceptable salt thereof
wherein,
R¹, R² and R^{a}, which may be the same or different, are each independently hydrogen or (C₁-C₄)alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

A representative TRPA1 antagonist useful in the context of the invention is Compound 91:

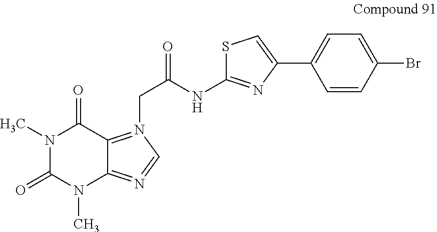

The Compound 91 may be prepared, for example, by following the process provided for the preparation of similar compounds in PCT publication No. WO2007073505.

In another aspect, TRPA1 antagonists useful in the context of the invention are selected from those compounds generically or specifically disclosed in WO2011114184. Accordingly, a TRPA1 antagonist useful in the context of the invention has the formula (IX):

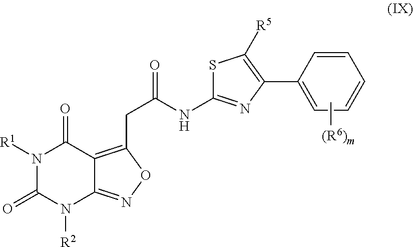

or a pharmaceutically-acceptable salt thereof

wherein at each occurrence, R¹ and R² are independently selected from hydrogen or substituted or unsubstituted alkyl;

at each occurrence, R⁵ is selected from hydrogen, halogen or substituted or unsubstituted alkyl;

at each occurrence, R⁶ is selected from hydrogen, cyano, nitro, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl.

A representative TRPA1 antagonist useful in the methods of the invention is mentioned below:

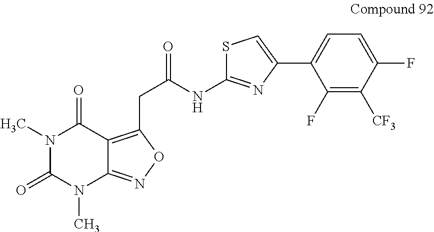 [SMILES: Cn1c(=O)c2c(CC(=O)Nc3nc(-c4ccc(F)c(C(F)(F)F)c4F)cs3)onc2n(C)c1=O]

The preparation of above said compounds is described in WO2011114184.

In another aspect, TRPA1 antagonist useful in the context of the invention has the formula (X):

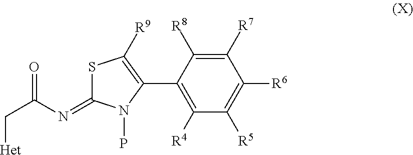

wherein, ‘Het’ is selected from groups consisting of

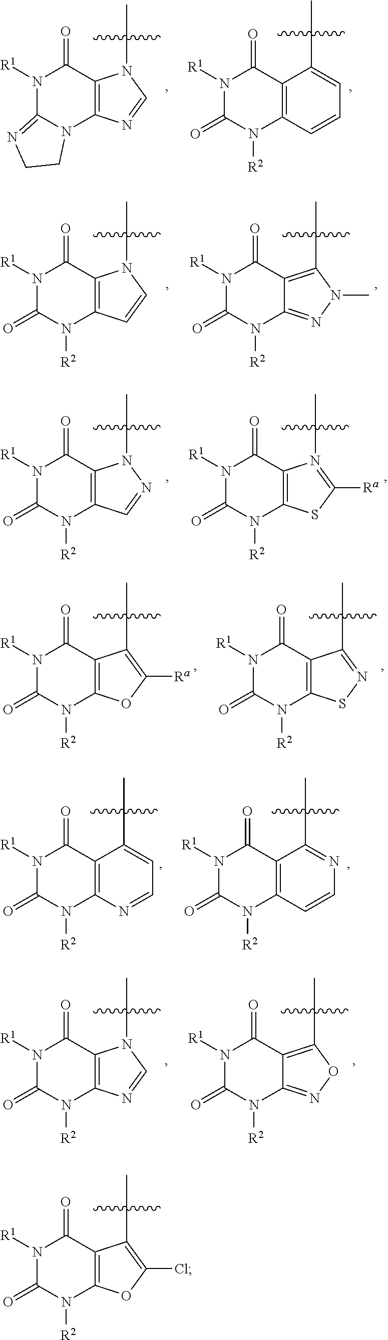

P is selected from

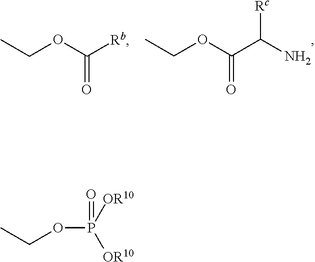

R¹, R² and R^{a}, which may be the same or different, are each independently hydrogen or (C₁-C₄) alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R^{b} and R^{c} independently selected from hydrogen, substituted or unsubstituted alkyl arylalkyl, amino acid and heterocyclic ring;

R¹⁰ is selected from hydrogen, alkyl, arylalkyl and pharmaceutically acceptable cation.

Few representative TRPA1 antagonists useful in the context of the invention are mentioned below:

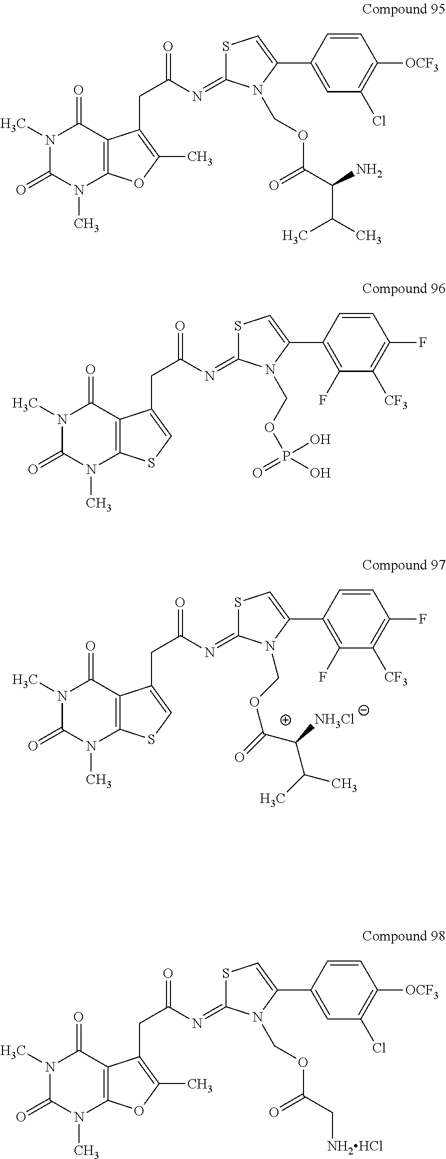

In another aspect, TRPA1 antagonists useful in the context of the invention are selected from those compounds generically or specifically disclosed in WO2011114184. Accordingly, TRPA1 antagonist useful in the context of the invention has the formula (XI):

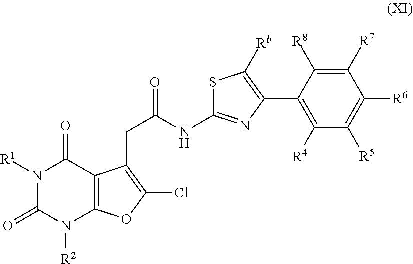

or a pharmaceutically acceptable salt thereof,

wherein, R¹, and R² are independently hydrogen or (C₁-C₄)alkyl; and

R⁴, R⁵, R⁶, R⁷, R⁸ and R⁹, which may be same or different, are each independently selected from halogen haloalkyl, dialkylamino, and haloalkoxy.

Few representative TRPA1 antagonists useful in the context of the invention are mentioned below:

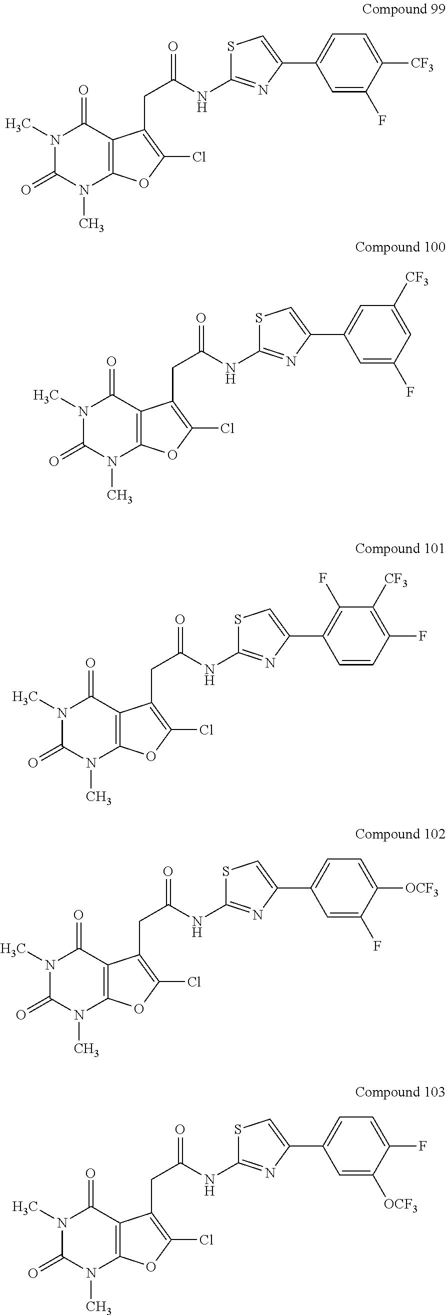 [SMILES: Cn1c(=O)c2c(CC(=O)Nc3nc(-c4ccc(C(F)(F)F)c(F)c4)cs3)c(Cl)oc2n(C)c1=O ; Cn1c(=O)c2c(CC(=O)Nc3nc(-c4cc(F)cc(C(F)(F)F)c4)cs3)c(Cl)oc2n(C)c1=O ; Cn1c(=O)c2c(CC(=O)Nc3nc(-c4ccc(F)c(C(F)(F)F)c4F)cs3)c(Cl)oc2n(C)c1=O ; Cn1c(=O)c2c(CC(=O)Nc3nc(-c4ccc(OC(F)(F)F)c(F)c4)cs3)c(Cl)oc2n(C)c1=O ; Cn1c(=O)c2c(CC(=O)Nc3nc(-c4ccc(F)c(OC(F)(F)F)c4)cs3)c(Cl)oc2n(C)c1=O]

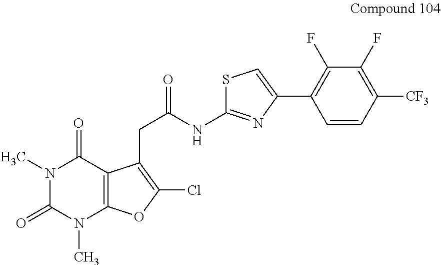 [SMILES: Cn1c(=O)c2c(CC(=O)Nc3nc(-c4ccc(C(F)(F)F)c(F)c4F)cs3)c(Cl)oc2n(C)c1=O]

The preparation of above said compounds is described in WO2011114184.

In an aspect, TRPA1 antagonists useful in the context of the invention, is selected from one of the following formulae: (XII) or (D)

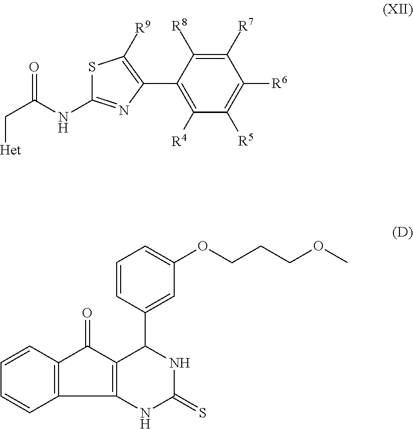

or a pharmaceutically-acceptable salt thereof, wherein, ‘Het’ is selected from the group consisting of

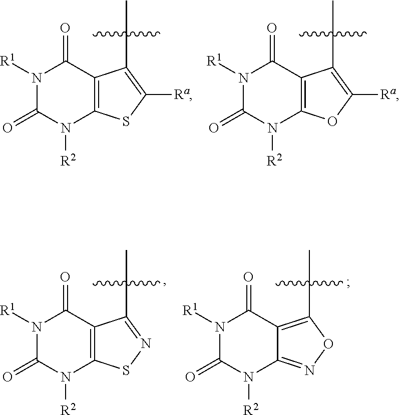

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists of the formula (XII) useful in the context of the invention are compound 52, compound 73 and compound 84 as described above.

The beta-2 agonist, as contemplated herein, includes salbutamol (INN: Albuterol), levosalbutamol, terbutaline, salmeterol, formoterol, metaproterenol, pirbuterol, bambuterol, procaterol, metaproterenol, fenoterol, bitolterol, indacaterol, vilanterol, arformoterol, olodaterol, abediterol, milveterol, bedoradrine, rirodrine clenbuterol, reproterol, PF-610355, GSK-597901, GSK-159802 and GSK-678007 or salts thereof. The salt may be present in the form of their isomers, polymorphs, and solvates, including hydrates, all of which are included in the scope of the invention. Preferably, the beta-2 agonist includes salbutamol, levosalbutamol, terbutaline, salmeterol, formoterol, arformoterol, indacaterol and bambuterol or salts thereof.

In an embodiment, the present invention relates to a pharmaceutical composition comprising synergistically effective amount of a TRPA1 antagonist having an IC₅₀ for inhibiting human TRPA1 receptor activity of less than 1 micromolar, and a beta-2 agonist. Preferably, the TRPA1 antagonist of the present invention has an IC₅₀ for inhibiting human TRPA1 receptor activity of less than 500 nanomolar, or more preferably less than 250 nanomolar, as measured by a method described herein.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

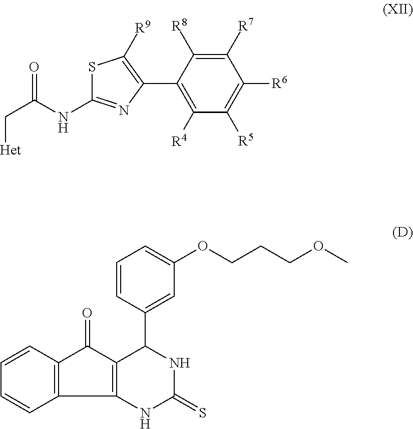

or a pharmaceutically-acceptable salt thereof, wherein, ‘Het’ is selected from the group consisting of

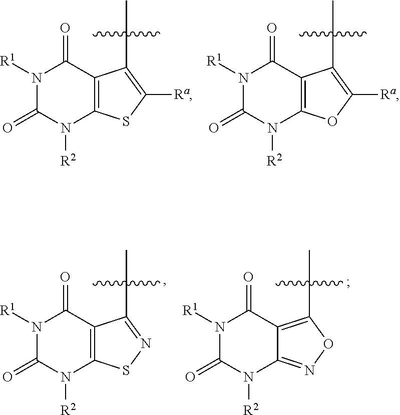

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and a beta-2 agonist.

In yet another embodiment, the present invention relates to a pharmaceutical composition comprising synergistically effective amount of a TRPA1 antagonist having structure of formula:

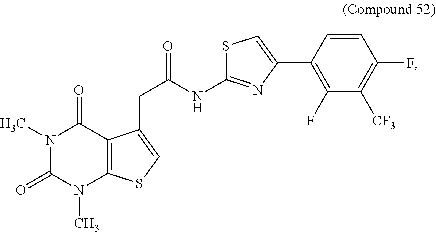 [SMILES: Cn1c(=O)c2c(CC(=O)Nc3nc(-c4ccc(F)c(C(F)(F)F)c4F)cs3)csc2n(C)c1=O]

and a beta-2 agonist.

In another embodiment, there is provided a pharmaceutical composition comprising synergistically effective amount of a TRPA1 antagonist having an IC₅₀ for inhibiting human TRPA1 receptor activity of less than 1 micromolar and a beta-2 agonist in a weight ratio ranging from about 1:0.0001 to about 1:4000.

In an embodiment, the present invention relates to a pharmaceutical composition comprising synergistically effective amount of a TRPA1 antagonist having structure of formula:

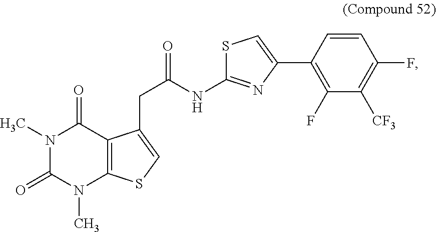 [SMILES: Cn1c(=O)c2c(CC(=O)Nc3nc(-c4ccc(F)c(C(F)(F)F)c4F)cs3)csc2n(C)c1=O]

and a beta-2 agonist selected from a group consisting of salbutamol, levosalbutamol, terbutaline, salmeterol, formoterol, arformoterol, indacaterol and bambuterol or salts thereof. In first aspect of this embodiment, the beta-2 agonist is salbutamol. In second aspect of this embodiment, the beta-2 agonist is levosalbutamol. In third aspect of this embodiment, the beta-2 agonist is terbutaline. In fourth aspect of this embodiment, the beta-2 agonist is salmeterol. In fifth aspect of this embodiment, the beta-2 agonist is formoterol. In sixth aspect of this embodiment, the beta-2 agonist is arformoterol. In seventh aspect of this embodiment, the beta-2 agonist is indacaterol. In another aspect of this embodiment, the pharmaceutical composition is a fixed dose combination.

The pharmaceutical composition of the present invention may be administered orally, nasally, intra-tracheally, parenterally, transdermally, transmucosal, inhalation or by any other route that a physician or a health-care provider may determine to be appropriate. Preferably, the route of administration is oral or by inhalation.

In another aspect of this embodiment, the composition is for oral administration and the TRPA1 antagonist and the beta-2 agonist are present in a weight ratio from about 1:0.01 to about 1:5.

In yet another aspect of this embodiment, the composition is for inhalation administration and the TRPA1 antagonist and the beta-2 agonist are present in a weight ratio from about 1:0.001 to about 1:300.

As contemplated herein, the active ingredients may be administered together in a single dosage form or they may be administered in different dosage forms. They may be administered at the same time or they may be administered either close in time or remotely, such as, where one drug is administered in the morning and the second drug is administered in the evening. The combination may be used prophylactically or after the onset of symptoms has occurred.

In a preferred embodiment, both the active ingredients i.e., TRPA1 antagonist and the beta-2 agonist are formulated as a pharmaceutical composition suitable for administration by the same route (e.g., both the actives by oral or inhalation route), or by different routes (e.g., one active by oral and the other active by inhalation route).

The pharmaceutical compositions for oral administration may be in conventional forms, for example, tablets, capsules, granules (synonymously, “beads” or “particles” or “pellets”), suspensions, emulsions, powders, dry syrups, and the like. The capsules may contain granule/pellet/particle/mini-tablets/mini-capsules containing the active ingredients. The amount of the active agent that may be incorporated in the pharmaceutical composition may range from about 1% w/w to about 98% w/w or from about 5% w/w to about 90% w/w.

The pharmaceutical compositions for parenteral administration include but are not limited to solutions/suspension/emulsion for intravenous, subcutaneous or intramuscular injection/infusion, and implants. The pharmaceutical compositions for transdermal or transmucosal administration include but are not limited to patches, gels, creams, ointments and the like.

As set forth above, the pharmaceutical composition includes at least one pharmaceutically acceptable excipient, which includes but is not limited to one or more of the following; diluents, glidants and lubricants, preservatives, buffering agents, chelating agents, polymers, gelling agents/viscosifying agents, surfactants, solvents and the like.

In an embodiment, the present invention provides a process for the preparing a pharmaceutical composition comprising TRPA1 antagonist and a beta-2 agonist and a pharmaceutically acceptable excipient, wherein the composition is in the form of a fixed dose combination formulation. The process comprises admixing TRPA1 antagonist with the beta-2 agonist. Alternately, the process comprises formulating TRPA1 antagonist and the beta-2 agonist in such a way that they are not in intimate contact with each other.

In another embodiment, the invention relates to a process for preparing a pharmaceutical composition comprising TRPA1 antagonist, a beta-2 agonist and a pharmaceutically acceptable excipient, wherein the composition is in the form of kit comprising separate formulations of TRPA1 antagonist and the beta-2 agonist.

The process for making the pharmaceutical composition may for example include, (1) granulating either or both the active ingredients, combined or separately, along with pharmaceutically acceptable carriers so as to obtain granulate, and (2) converting the granulate into suitable dosage forms for oral administration. The typical processes involved in the preparation of the pharmaceutical combinations include various unit operations such as mixing, sifting, solubilizing, dispersing, granulating, lubricating, compressing, coating, and the like. These processes, as contemplated by a person skilled in the formulation art, have been incorporated herein for preparing the pharmaceutical composition of the present invention.

### Methods of Treatment

Asthma and COPD are major chronic diseases related to airway obstruction. The Global Initiative for Chronic Obstructive Lung Disease provides guidelines for the distinction between asthma and COPD. Asthma is believed to be a chronic inflammatory disease wherein the airflow limitation is more or less reversible while it is more or less irreversible in case of COPD. Asthma among other things is believed to be triggered by inhalation of sensitizing agents (like allergens) unlike noxious agents (like particles and certain gases) in case of COPD. Though both are believed to have an inflammatory component, the inflammation in asthma is believed to be mostly eosinophilic and CD-4 driven, while it is believed to be mostly neutrophilic and CD-8 driven in COPD.

Asthma is clinically classified according to the frequency of symptoms, forced expiratory volume in 1 second (FEV₁), peak expiratory flow rate and severity (e.g., acute, intermittent, mild persistent, moderate persistent, and severe persistent). Asthma may also be classified as allergic (extrinsic) or non-allergic (intrinsic), based on whether symptoms are precipitated by allergens or not. Asthma can also be categorized according to following types viz., nocturnal asthma, bronchial asthma, exercise induced asthma, occupational asthma, seasonal asthma, silent asthma, and cough variant asthma.

COPD, also known as chronic obstructive lung disease (COLD), chronic obstructive airway disease (COAD), or chronic obstructive respiratory disease (CORD), is believed to be the co-occurrence of chronic bronchitis (characterized by a long-term cough with mucus) and emphysema (characterized by destruction of the lungs over time), a pair of commonly co-existing diseases of the lungs in which the airways become narrowed. This leads to a limitation of the flow of air to and from the lungs, causing shortness of breath. An acute exacerbation of COPD is a sudden worsening of COPD symptoms (shortness of breath, quantity and color of phlegm) that typically lasts for several days and is believed to be triggered by an infection with bacteria or viruses or by environmental pollutants. Based on the FEV₁ values, COPD can be classified as mild, moderate, severe and very severe.

Various classes of drugs are currently being used for the treatment and/or prophylaxis of respiratory disorders like asthma and COPD. Some of the classes of such drugs are leukotriene receptor antagonists, antihistamines, beta-2 agonists, anticholinergic agents and corticosteroids.

Beta-2 agonists are commonly used for the control of respiratory disorders like asthma to a large extent and COPD to a lesser extent. The beta-2 agonists are believed to selectively bind to the beta-2 adrenergic receptors that are found in cell membrane of airway cells like airway smooth muscle, airway epithelial cells, mast cells, endothelium and the vascular smooth muscle. The binding of the beta-2 agonist to the adrenergic receptor in airway smooth muscle is believed to trigger a signaling cascade that eventually leads to airway smooth muscle relaxation. The binding of the beta-2 agonist to the beta-2 adrenergic receptor causes the activation of the G-protein coupled to the receptor, which in turn, stimulates the adenylyl cyclase, and such stimulation of adenylyl cyclase is then believed to bring about the airway smooth muscle relaxation. Beta-2 agonists are also believed to protect asthmatics against bronchoconstrictor stimuli. Thus, beta-2 agonists are effective in the long-term management of respiratory disorders, such as asthma.

Thus, it is believed that though the therapeutic outcomes of these two classes of drugs, the TRPA1 antagonists and the beta-2 agonists are similar to some extent, the mechanism of actions may vary to a good extent and thus the therapeutic effect of their combination in the treatment of respiratory disorders is highly unpredictable. Particularly, the therapeutic effect of the combination of TRPA1 antagonist and a beta-2 agonist is highly unpredictable.

The inventors of the present invention have surprisingly found that a pharmaceutical composition comprising TRPA1 antagonist and a beta-2 agonist are more effective in the treatment of respiratory disorders, and provide better therapeutic value when compared to both the actives alone (when administered individually) for the treatment of respiratory disorders.

In an embodiment, the present invention relates to a method of treating a respiratory disorder in a subject, said method comprising administering to the subject the pharmaceutical composition comprising synergistically effective amount of a TRPA1 antagonist having an IC₅₀ for inhibiting human TRPA1 receptor activity of less than 1 micromolar and a beta-2 agonist. In an aspect of this embodiment, the TRPA1 antagonist has an IC₅₀ for inhibiting human TRPA1 receptor activity of less than 1 micromolar having structure of formulae: (XII) or (D)

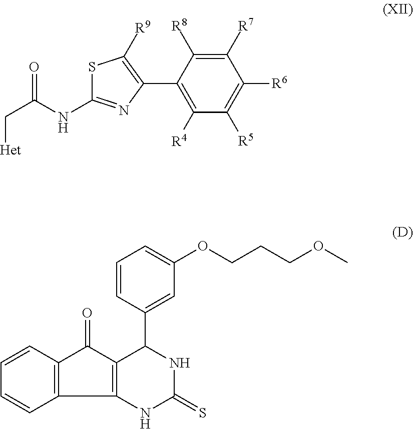

or a pharmaceutically-acceptable salt thereof, wherein, ‘Het’ is selected from the group consisting of

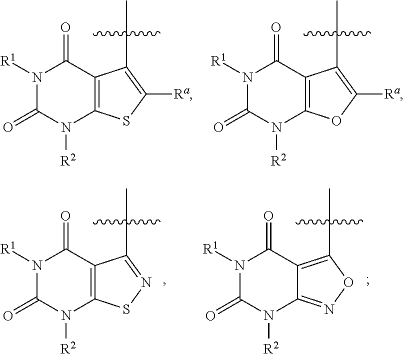

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a method of treating a respiratory disorder in a subject, said method comprising administering the subject a pharmaceutical composition comprising synergistically effective amount of a TRPA1 antagonist having an IC₅₀ for inhibiting human TRPA1 receptor activity of less than 1 micromolar and beta-2 agonist selected from a group consisting of salbutamol, levosalbutamol, terbutaline, salmeterol, formoterol, arformoterol, indacaterol and bambuterol or salts thereof.

In a further embodiment, the present invention relates to use of synergistically effective amount of a TRPA1 antagonist having an IC₅₀ for inhibiting human TRPA1 receptor activity of less than 1 micromolar and a beta-2 agonist in the preparation of a pharmaceutical composition of the present invention for the treatment of a respiratory disorder in a subject. In an aspect of this embodiment, the TRPA1 antagonist has an IC₅₀ for inhibiting human TRPA1 receptor activity of less than 1 micromolar having structure of formulae: (XII) or (D)

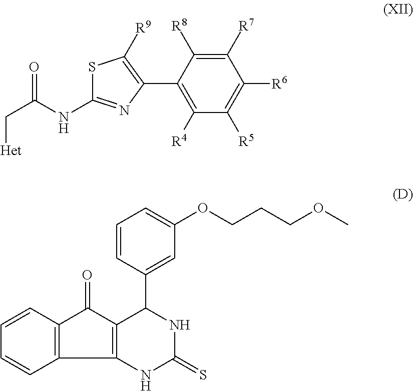

or a pharmaceutically-acceptable salt thereof, wherein, ‘Het’ is selected from the group consisting of

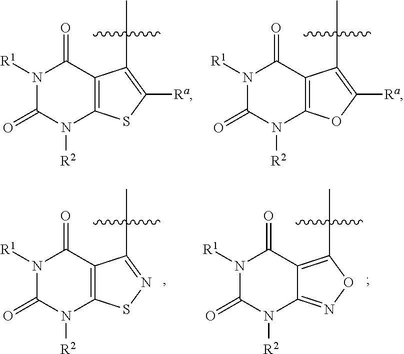

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a pharmaceutical composition comprising synergistically effective amount of a TRPA1 antagonist having an IC₅₀ for inhibiting human TRPA1 receptor activity of less than 1 micromolar and a beta-2 agonist for the treatment of a respiratory disorder in a subject.

In an embodiment, the present invention relates to a method of treating a respiratory disorder in a subject, said method comprising administering to the subject the pharmaceutical composition comprising synergistically effective amount of a TRPA1 antagonist having structure of formula:

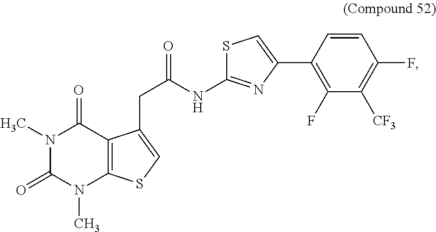 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof.

In an embodiment, the present invention relates to a method of treating a respiratory disorder by improving lung function in a subject, said method comprising administering to the subject the pharmaceutical composition comprising synergistically effective amount of a TRPA1 antagonist having structure of formula:

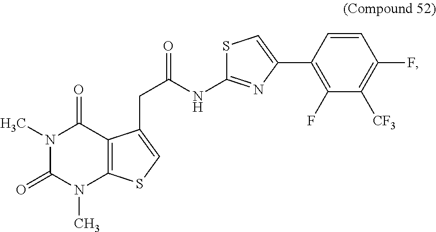 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof. In another aspect of this embodiment, the respiratory disorder is asthma.

In an embodiment, the present invention relates to a method of improving lung function in a subject, said method comprising administering to the subject the pharmaceutical composition comprising synergistically effective amount of a TRPA1 antagonist having structure of formula:

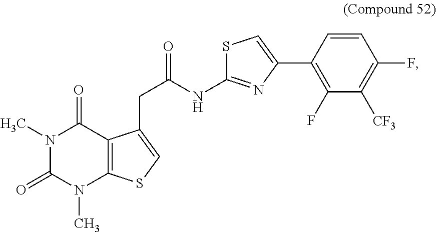 [SMILES: Cn1c(=O)c2c(CC(=O)Nc3nc(-c4ccc(F)c(C(F)(F)F)c4F)cs3)csc2n(C)c1=O]

and a beta-2 agonist. In an aspect of this embodiment, the beta-2 agonist selected from a group consisting of salbutamol, levosalbutamol, terbutaline, salmeterol, formoterol, arformoterol, indacaterol and bambuterol or salts thereof.

In another embodiment, the present invention relates to use of synergistically effective amount of a TRPA1 antagonist having structure of formula:

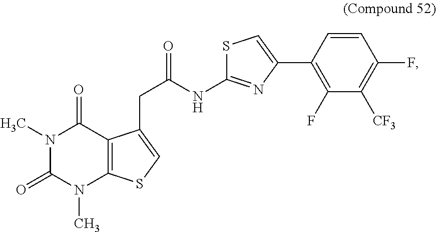 [SMILES: Cn1c(=O)c2c(CC(=O)Nc3nc(-c4ccc(F)c(C(F)(F)F)c4F)cs3)csc2n(C)c1=O]

and beta-2 agonist in the preparation of a pharmaceutical composition of the present invention for the treatment of a respiratory disorder in a subject. In an aspect of this embodiment, the beta-2 agonist selected from a group consisting of salbutamol, levosalbutamol, terbutaline, salmeterol, formoterol, arformoterol, indacaterol and bambuterol or salts thereof.

In a further embodiment, the present invention relates to a pharmaceutical composition comprising synergistically effective amount of a TRPA1 antagonist having structure of formula:

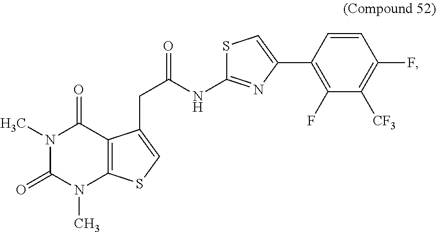 [SMILES: Cn1c(=O)c2c(CC(=O)Nc3nc(-c4ccc(F)c(C(F)(F)F)c4F)cs3)csc2n(C)c1=O]

and a beta-2 agonist for the treatment of a respiratory disorder in a subject.

The therapeutically effective amount of TRPA1 antagonist to be administered per day ranges from about 10 mcg/kg to about 20 mg/kg, and preferably from about 50 mcg/kg to about 15 mg/kg. The therapeutically effective amount of salmeterol or its salt to be administered per day ranges from about 10 mcg to about 1 mg, and preferably from about 20 mcg to about 500 mcg and more preferably from about 50 mcg to about 100 mcg. Preferably, the discrete dosage strengths of salmeterol or its salt to be administered per day are 50 mcg and 100 mcg.

The therapeutically effective amount of formoterol or its salt to be administered per day ranges from about 1 mcg to about 100 mcg, and preferably from about 5 mcg to about 50 mcg and more preferably from about 10 mcg to about 25 mcg. Preferably, the discrete dosage strengths of formoterol or its salt to be administered per day are 12 mcg and 24 mcg.

The therapeutically effective amount of indacaterol or its salt to be administered per day ranges from about 50 mcg to about 500 mcg; and preferably from about 100 mcg to about 400 mcg and more preferably from about 150 mcg to about 300 mcg. Preferably, the discrete dosage strengths of indacaterol or its salt to be administered per day are 150 mcg and 300 mcg.

The therapeutically effective amount of salbutamol or its salt to be administered per day ranges from about 0.01 mg to about 30 mg and more preferably from about 0.05 mg to about 20 mg. Preferably, the discrete dosage strengths of salbutamol or its salt to be administered per day are 1 mg, 2 mg, 3 mg, 4 mg, 6 mg, 8 mg, 10 mg, 12 mg and 16 mg.

The therapeutically effective amount of levosalbutamol or its salt to be administered per day ranges from about 0.1 mg to about 5 mg and preferably from about 0.2 mg to about 4.5 mg and more preferably from about 0.3 mg to about 4 mg. Preferably, the discrete dosage strengths of levosalbutamol or its salt to be administered per day are 0.31 mg, 0.62 mg, 0.63 mg, 0.93 mg, 1.25 mg, 1.26 mg, 1.89 mg, 2.5 mg and 3.75 mg.

The therapeutically effective amount of terbutaline or its salt to be administered per day ranges from about 0.1 mg to about 1 mg and preferably from about 0.2 mg to about 0.75 mg and more preferably from about 0.25 mg to about 0.5 mg. Preferably, the discrete dosage strengths of terbutaline or its salt to be administered per day ranges are 0.25 mg and 0.5 mg.

The optimal dose of the active ingredient or the combination of the active ingredients can vary as a function of the severity of disease, route of administration, composition type, the patient body weight, the age and the general state of mind of the patient, and the response to behavior to the active ingredient or the combination of the active ingredients.

In the pharmaceutical composition as described herein, the active ingredient may be in the form of a single dosage form (i.e., fixed-dose formulation in which both the active ingredients are present together) or they may be divided doses, formulated separately, each in its individual dosage forms but as part of the same therapeutic treatment, program or regimen, either once daily or two/three/four times a day.

Alternately, the invention relates to a pharmaceutical composition wherein the composition is in the form of kit comprising separate formulations of TRPA1 antagonist and the beta-2 agonist. The separate formulations are to be administered by same or different routes, either separately, simultaneously, or sequentially, where the sequential administration is close in time or remote in time. For sequential administration, the period of time may be in the range from 10 min to 12 hours.

Various animal models have been used for the evaluation of the therapeutic efficacy of drug candidates for respiratory disorders like asthma and COPD. For example, commonly used strategy for evaluation of drug candidates in asthma is the allergen sensitization and challenge method. The commonly used such model is the ovalbumin (OVA) sensitization and challenge in laboratory animals. Another model that can be used is the methacholine challenge test by using invasive whole body plethysmograph.

A commonly used model for evaluation of drug candidates in COPD involves the chronic exposure of the animal to SO₂ or tobacco/cigarette smoke. The model is believed to generate sloughing of epithelial cells, increase in the mucus secretions, increase in the polymorphonuclear cells and pulmonary resistance, and increase in the airway hyper-responsiveness (in rats).

Another model that can be used for evaluation of drug candidates in COPD involves the exposure of animals (e.g., rats) to lipopolysaccharide (LPS). The exposure to LPS is believed to result in the influx of neutrophils in the lungs, a condition that is believed to be one of the characteristics of COPD.

It will be understood that various modifications may be made to the embodiments disclosed herein. Therefore the above description should not be construed as limiting, but merely as exemplifications of preferred embodiments. Other arrangements and methods may be implemented by those skilled in the art without departing from the scope and spirit of this invention.

The following examples are provided to enable one skilled in the art to practice the invention and are merely illustrative of the invention. The examples should not be read as limiting the scope of the invention.

### EXAMPLES

### Example 1

### Determination of IC₅₀ of TRPA1 Antagonists

The human IC₅₀ values were measured by the following method:

The inhibition of TRPA1 receptor activation is measured as inhibition of allylisothiocyanate (AITC) induced cellular uptake of radioactive calcium.

Test compound solution is prepared in a suitable solvent.

Human TRPA1 expressing CHO cells are grown in suitable medium. Cells are treated with test compounds followed by addition of AITC.

Cells are washed, lysed and the radioactivity in the lysate is measured in Packard Top count after addition of liquid scintillant.

The concentration response curves for compounds are plotted as a % of maximal response obtained in the absence of test antagonist, and the IC₅₀ values are calculated from such concentration response curve by nonlinear regression analysis using GraphPad PRISM software.

TABLE 1
TRPA1 antagonists having a human IC₅₀ for inhibiting human TRPA1
receptor activity of less than 1 micromolar.

| Compound No | hTRPA1 IC₅₀ values | |
|---|---|---|
| 1 | 920.9 | nM |
| 2 | 381.8 | nM |
| 3 | 73.35 | nM |
| 4 | 98.32 | nM |
| 5 | 66.28 | nM |
| 6 | 97.42 | nM |
| 7 | 47.37 | nM |
| 8 | 55.02 | nM |
| 9 | 102.5 | nM |
| 10 | 46.74 | nM |
| 11 | 46.27 | nM |
| 12 | 51.68 | nM |
| 13 | 48.21 | nM |
| 14 | 60.42 | nM |
| 15 | 53.57 | nM |
| 16 | 58.94 | nM |
| 17 | 56.02 | nM |
| 18 | 13.38 | nM |
| 19 | 26.13 | nM |
| 20 | 20.09 | nM |
| 21 | 48.18 | nM |
| 22 | 79.77 | nM |
| 23 | 43.93 | nM |
| 24 | 138.1 | nM |
| 25 | 58.55 | nM |
| 26 | 47.91 | nM |
| 27 | 65.45 | nM |
| 28 | 6.49 | nM |
| 29 | 11.38 | nM |
| 30 | 34.03 | nM |
| 31 | 17.3 | nM |
| 32 | 5.96 | nM |
| 33 | 5.37 | nM |
| 34 | 38.46 | nM |
| 35 | 18.05 | nM |
| 36 | 49.92 | nM |
| 37 | 12.26 | nM |
| 38 | 15.92 | nM |
| 39 | 26.56 | nM |
| 40 | 22.82 | nM |
| 41 | 11.04 | nM |
| 42 | 11.38 | nM |
| 43 | 18.37 | nM |
| 44 | 8.36 | nM |
| 45 | 26.39 | nM |
| 46 | 41.31 | nM |
| 47 | 33.61 | nM |
| 48 | 18.12 | nM |
| 49 | 3.98 | nM |
| 50 | 16.73 | nM |
| 51 | 4.84 | nM |
| 52 | 2.49 | nM |
| 53 | 18.20 | nM |
| 54 | 17.74 | nM |
| 55 | 2.15 | nM |
| 56 | 3.38 | nM |
| 57 | 1.45 | nM |
| 58 | 11.88 | nM |
| 59 | 2.21 | nM |
| 60 | 3.54 | nM |
| 61 | 2.93 | nM |
| 62 | 1.68 | nM |
| 63 | 9.04 | nM |
| 64 | 4.52 | nM |
| 65 | 6.65 | nM |
| 66 | 3.63 | nM |
| 67 | 13.59 | nM |
| 68 | 4.84 | nM |
| 69 | 7.10 | nM |
| 70 | 12.57 | nM |
| 71 | 3.18 | nM |
| 72 | 4.16 | nM |
| 73 | 8.54 | nM |
| 74 | 5.29 | nM |
| 75 | 3.34 | nM |
| 76 | 4.02 | nM |
| 77 | 5.60 | nM |
| 78 | 10.57 | nM |
| 79 | 5.29 | nM |
| 80 | 6.28 | nM |
| 81 | 6.74 | nM |
| 82 | 8.04 | nM |
| 83 | 4.40 | nM |
| 84 | 5.35 | nM |
| 85 | 8.92 | nM |
| 86 | 6.91 | nM |
| 87 | 19.32 | nM |
| 88 | 11.45 | nM |
| 89 | 98.44 | nM |
| 90 | 5.61 | nM |
| 91 | 451.4 | nM |
| 92 | 17.08 | nM |
| 95 | 88.50 | nM |
| 96 | 559.3 | nM |
| 97 | 21.91 | nM |
| 98 | 54.29 | nM |
| 99 | 5.06 | nM |
| 100 | 5.15 | nM |
| 101 | 10.10 | nM |
| 102 | 7.67 | nM |
| 103 | 27.41 | nM |
| 104 | 7.58 | nM |

### Example 2

### Animal Studies for the Combination of TRPA1 Antagonist and Salmeterol

The effect of the treatments (alone and in combination) on methacholine induced bronchoconstriction (airway hyperresponsiveness) in ovalbumin (Ova) sensitized female BALB/c mice was studied. Female BALB/c mice were sensitized on day 0 and 7 with 50 mcg ovalbumin and 4 mg alum given intraperitonially (i.p.). Mice were challenged with 3% aerosolized ovalbumin from Day 11-13 following sensitization. Sensitized mice were randomly assigned to different treatment groups according to Table 2. Test compounds were triturated with 2 drops of Tween-80 and volume was made up with 0.5% methyl cellulose (MC) solution for oral administration. Animals were administered Compound 52 orally 24 hours before l^{st} ovalbumin challenge and 2 hours before ovalbumin challenge from Day-11 to 13.

On day 14, animals were pretreated with the compounds (2 hours pretreatment for Compound 52 and 1 hour pretreatment for salmeterol administered orally) and challenged with increasing concentrations of methacholine (Mch) in Buxco Whole Body Plethysmography chamber and AHR was determined.

TABLE 2

| | No. of | Route of | |
|---|---|---|---|
| Group | animals | administration | Treatment/Exposure |
| A | 10 | p.o. | Saline Control |
| B | 10 | | Vehicle (p.o.) treated/Ovalbumin |
| | | | sensitized/Ovalbumin challenged |
| | | | (OVA/Vehicle/Mch) |
| C | 7 | | Compound 52 treated/Ovalbumin |
| | | | sensitized/Ovalbumin challenged |
| | | | (Ova + Compound 52/Mch) |
| D | 7 | | Salmeterol (p.o.) treated/Ovalbumin |
| | | | sensitized/Ovalbumin challenged |
| | | | (Ova + Salmeterol/Mch) |
| E | 8 | | (Compound 52 + Salmeterol) treated/ |
| | | | Ovalbumin sensitized/Ovalbumin |
| | | | challenged (OVA/Combination/Mch) |

Airway responsiveness to methacholine was assessed in conscious, unrestrained mice by whole-body plethysmography (Buxco, Wilmington, N.C.) approximately 24 hours after last ovalbumin challenge. Briefly, mice were placed in the main chamber (animal chamber) of the plethysmograph, and the pressure differences (termed the box pressure signal) between this chamber and a reference chamber integral to the main chamber were measured with a differential pressure transducer connected to the amplifier. From the box pressure signal, the phases of the respiratory cycle, tidal volumes, and Penh can be calculated.

Penh is a dimensionless value that represents a function of the proportion of maximal expiratory to maximal inspiratory box pressure signals and of the timing of expiration. According to the manufacturer's instructions, Penh was calculated as follows:

Penh=(Te−Tr)/Tr (defined as “pause”)×(PEF/PIP),

where, Te—expiratory time (seconds);
Tr—relaxation time (seconds), defined as the time of pressure decay to 30% of the total expiratory pressure signal (area under the box pressure signal at expiration);
PEF—Peak expiratory flow (ml/second); and
PIP—Peak inspiratory flow (ml/second).
Combination of Compound 52 and salmeterol (Group E) produced significantly superior inhibition of MCh induced bronchoconstriction (penh) as compared to the individual activity of both treatments (Group C and Group D). The results are shown in FIG. 1.

### Example 3

### Animal Studies for the Combination of TRPA1 Antagonist and Formoterol

Female BALB/c mice were sensitized on day 0 and 7 with 50 mcg ovalbumin and 4 mg alum given intraperitonealy. Mice were challenged with 3% aerosolized ovalbumin from Day 11-13 following sensitization. Sensitized mice were randomly assigned to different treatment groups. Test compounds were triturated with 2 drops of Tween-80 and volume was made up with 0.5% methyl cellulose (MC) solution for oral administration. Animals were administered Compound 52 orally 24 hours before 1^{st} ovalbumin challenge and 2 hours before ovalbumin challenge from Day-11 to 13. Animals were administered Formoterol orally 24 hours before 1^{st} ovalbumin challenge and 1 hour before ovalbumin challenge from Day-11 to 13. On day 14, animals were pretreated with the compounds (2 hours pretreatment for Compound 52 and 1 hour pretreatment for Formoterol) and challenged with increasing concentrations of methacholine in Buxco Whole Body Plethysmography chamber and AHR was determined. Statistical analysis was performed using One Way ANOVA followed by Dunnett's multiple comparisons with the help of Graph Pad Prism software. Statistical significance was set at p<0.05.

Animals were assigned to one of the following 5 groups.

TABLE 3

| | | | Dose | | |
|---|---|---|---|---|---|
| | | | Formoterol | Compound 52 | |
| Groups | Treatment | n | (mg/kg, p.o.) | (mg/kg, p.o.) | Exposure to |
| 1 | Saline control | 8 | — | — | Saline |
| 2 | Vehicle | 6 | — | — | MCh |
| | (OVA/Vehicle/Mch) | | | | |
| 3 | Ova + Compound 52 | 6 | — | 1 | |
| | (OVA/Compound 52/ | | | | |
| | Mch) | | | | |
| 4 | Ova + Formoterol | 6 | 0.1 | — | |
| | (OVA/Formoterol/Mch) | | | | |
| 5 | Combination | 6 | 0.1 | 1 | |
| | (OVA/Combination/Mch) | | | | |

Airway responsiveness to methacholine was assessed in conscious, unrestrained mice by whole-body plethysmography (Buxco, Wilmington, N.C.) approximately 24 hour after last ovalbumin challenge. Briefly, mice were placed in the main chamber (animal chamber) of the plethysmograph, and the pressure differences (termed the box pressure signal) between this chamber and a reference chamber integral to the main chamber were measured with a differential pressure transducer connected to the amplifier. From the box pressure signal, the phases of the respiratory cycle, tidal volumes, and Penh can be calculated. Penh is a dimensionless value that represents a function of the proportion of maximal expiratory to maximal inspiratory box pressure signals and of the timing of expiration. Penh was calculated as given in Example 2.

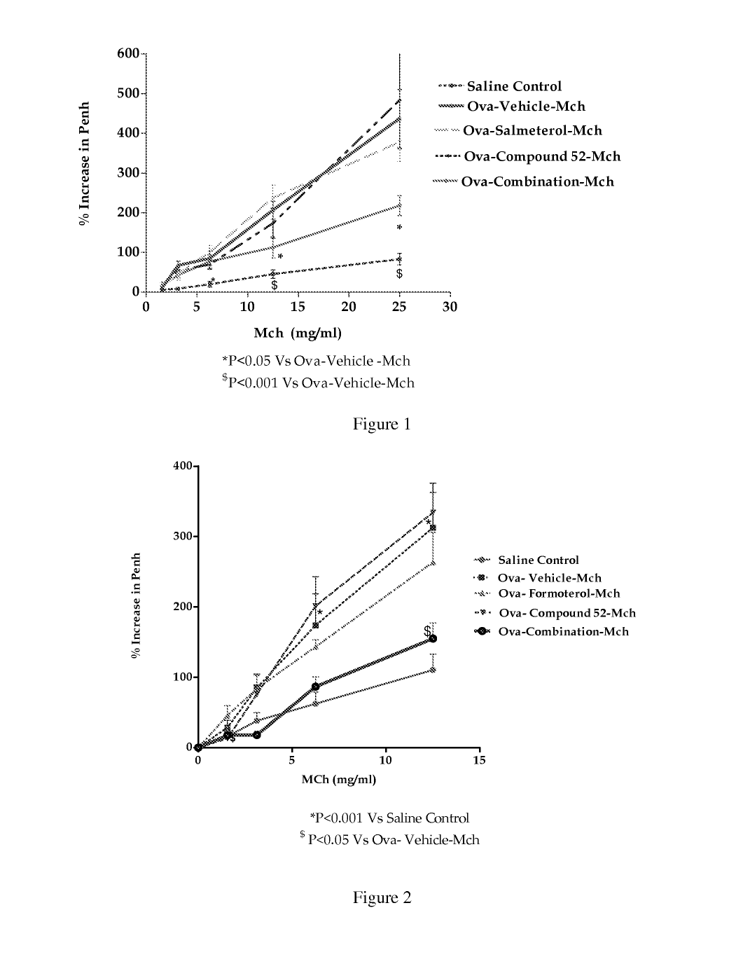

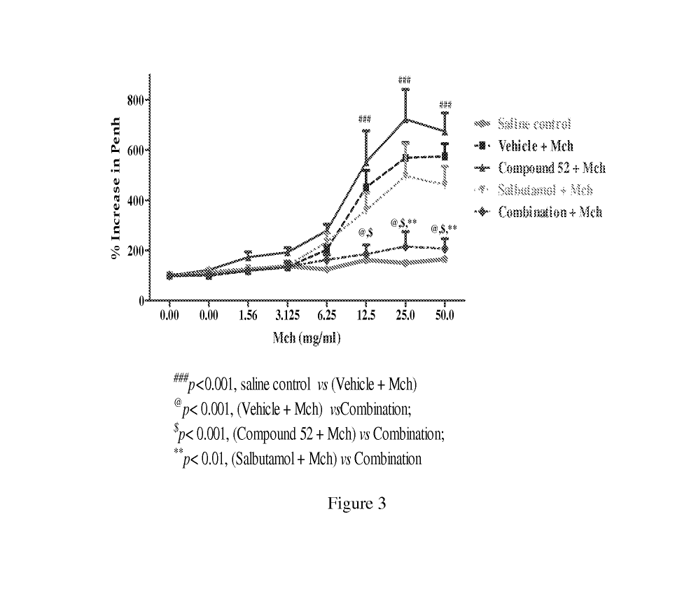

Compound 52 in combination with Formoterol (Group 5) showed significant synergy in inhibition of methacholine induced airway hyperresponsiveness as compared to the individual activity of both treatments (Group 3 and Group 4). The results are shown in FIG. 2.

### Example 4

### Animal Studies for the Combination of TRPA1 Antagonist and Salbutamol

BALB/c mice weighing 25-30 gm were grouped as mentioned in Table 1. Compound 52 (3 mg/kg) and salbutamol (1 mg/kg/10 ml) were administered by oral route. One hour post administration of vehicle or test compounds, the animals were challenged to MCh aerosol (1.56, 3.125, 6.25, 12.5, 25 and 50 mg/ml in cumulative fashion) using Buxco nebulizer in noninvasive whole body plethysmograph and the exposure period was kept at 150 seconds for each ascending dose of MCh. Animals of saline control group were given saline exposure under similar conditions. MCh induced bronchoconstriction at each of cumulative doses was recorded for a period of 150 seconds in the form of enhanced pause (Penh). Effect of respective treatments on MCh induced bronchoconstriction was calculated as percent inhibition in penh with respect to vehicle (MCh) control group taking into consideration the saline control group.

Animals were assigned to one of the following 5 groups.

TABLE 4

| | | | Dose | | |
|---|---|---|---|---|---|
| | | | Salbutamol | Compound 52 | Exposure |
| Groups | Treatment | n | (mg/kg, p.o.) | (mg/kg, p.o.) | to |
| 1 | Saline control | 7 | — | — | Saline |
| 2 | MCh control | 12 | — | — | MCh |
| | (Vehicle + | | | | |
| | Mch) | | | | |
| 3 | Compound 52 | 7 | — | 3 | |
| | (Compound | | | | |
| | 52 + Mch) | | | | |
| 4 | Salbutamol | 6 | 1 | — | |
| | (Salbutamol + | | | | |
| | Mch) | | | | |
| 5 | Combination | 7 | 1 | 3 | |
| | (Combination + | | | | |
| | Mch) | | | | |

### Recording Procedure:

1. Baseline recording for the first 300 sec.
2. Saline exposure for 150 seconds followed by data recording for next 150 seconds.
3. MCh exposure (at 1.56, 3.125, 6.25, 12.5, 25 & 50 mg/ml) for 150 seconds at each dose, followed by 150 sec of data recording in cumulative fashion.

Statistical analysis was performed using two way ANOVA followed by Bonferroni test. Graph-pad Prism software was used for the analysis. Statistical significance was set at p<0.05.

Compound 52 in combination with Salbutamol (Group 5) showed significant synergy in inhibition of MCh induced bronchoconstriction (Penh) as compared to the individual activity of both treatments (Group 3 and Group 4). The results are shown in FIG. 3.

Although the invention herein has been described with reference to particular embodiments, it is to be understood that these embodiments are merely illustrative of the principles and application of the present invention. It is therefore to be understood that numerous modifications may be made to the illustrative embodiments of the present invention as described.

All publications, patents, and patent applications cited in this application are herein incorporated by reference to the same extent as if each individual publication, patent, or patent application was specifically and individually indicated to be incorporated herein by reference.

1.-35. (canceled)

36. A pharmaceutical composition comprising synergistically effective amount of a TRPA1 antagonist having an IC₅₀ for inhibiting human TRPA1 receptor activity of less than 1 micromolar, and a beta-2 agonist selected from the group consisting of salmeterol, salbutamol, formoterol, indacaterol, olodaterol and vilanterol or a salt thereof.

37. The pharmaceutical composition according to claim 36, wherein the TRPA1 antagonist has an IC₅₀ for inhibiting human TRPA1 receptor activity of less than 500 nanomolar.

38. The pharmaceutical composition according to claim 37, wherein the TRPA1 antagonist has an IC₅₀ for inhibiting human TRPA1 receptor activity of less than 250 nanomolar.

39. The pharmaceutical composition according to claim 36, wherein the TRPA1 antagonist and the beta-2 agonist are present in a weight ratio from about 1:0.0001 to about 1:4000.

40. A pharmaceutical composition comprising synergistically effective amount of a TRPA1 antagonist having structure of formulae: (XII) or (D) 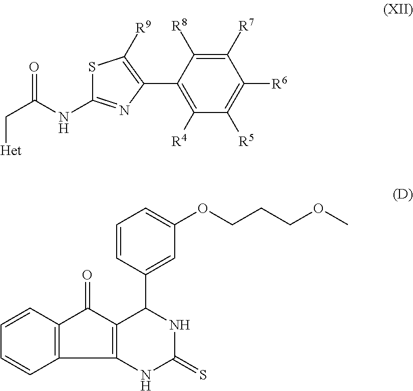
or a pharmaceutically-acceptable salt thereof, wherein,
‘Het’ is selected from the group consisting of 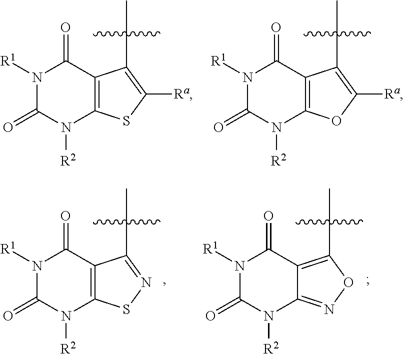
R¹, R² and R^{a}, which may be the same or different, are each independently hydrogen or (C1-C4) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl,
and a beta-2 agonist selected from the group consisting of salmeterol, salbutamol, formoterol, indacaterol, olodaterol and vilanterol or a salt thereof.

41. A pharmaceutical composition comprising synergistically effective amount of a TRPA1 antagonist having structure: 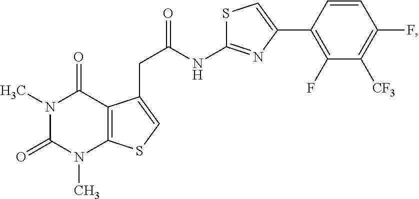 [SMILES: Cn1c(=O)c2c(CC(=O)Nc3nc(-c4ccc(F)c(C(F)(F)F)c4F)cs3)csc2n(C)c1=O]
and a beta-2 agonist selected from the group consisting of salmeterol, salbutamol, formoterol, indacaterol, olodaterol and vilanterol or a salts thereof.

42. The pharmaceutical composition according to claim 41, wherein the composition is a fixed dose combination.

43. The pharmaceutical composition according to claim 41, wherein the composition is for oral administration and the TRPA1 antagonist and the beta-2 agonist are present in a weight ratio from about 1:0.01 to about 1:5.

44. The pharmaceutical composition according to claim 41, wherein the composition is for inhalation administration and the TRPA1 antagonist and the beta-2 agonist are present in a weight ratio from about 1:0.001 to about 1:300.

45. A method of treating a respiratory disorder in a subject, said method comprising administering to the subject the pharmaceutical composition according to claim 41.

46. A method of treating a respiratory disorder by improving lung function in a subject, said method comprising administering to the subject the pharmaceutical composition according to claim 41.

47. A method of improving lung function in a subject, said method comprising administering to the subject the pharmaceutical composition according to claim 41.

48. The method according to claim 45, wherein the respiratory disorder is asthma.

49. The method according to claim 45, wherein the respiratory disorder is chronic obstructive pulmonary disease.

50. A pharmaceutical composition comprising synergistically effective amount of a TRPA1 antagonist having structure: 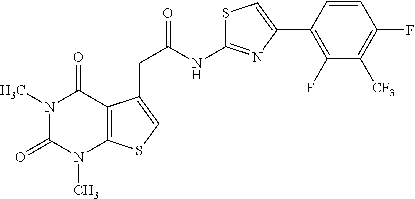 [SMILES: Cn1c(=O)c2c(CC(=O)Nc3nc(-c4ccc(F)c(C(F)(F)F)c4F)cs3)csc2n(C)c1=O]
a beta-2 agonist formoterol or its salt and a pharmaceutically acceptable excipient.

51. The pharmaceutical composition according to claim 50, wherein the composition is a fixed dose combination.

52. The pharmaceutical composition according to claim 50, wherein the composition is for oral administration and the TRPA1 antagonist and the beta-2 agonist are present in a weight ratio from about 1:0.01 to about 1:5.

53. The pharmaceutical composition according to claim 50, wherein the composition is for inhalation administration and the TRPA1 antagonist and the beta-2 agonist are present in a weight ratio from about 1:0.001 to about 1:300.

54. A method of treating a respiratory disorder in a subject, said method comprising administering to the subject the pharmaceutical composition according to claim 50.

55. A method of treating a respiratory disorder by improving lung function in a subject, said method comprising administering to the subject the pharmaceutical composition according to claim 50.

56. A method of improving lung function in a subject, said method comprising administering to the subject the pharmaceutical composition according to claim 50.

57. The method according to claim 54, wherein the respiratory disorder is chronic obstructive pulmonary disease.

58. A pharmaceutical composition comprising synergistically effective amount of a TRPA1 antagonist having structure: 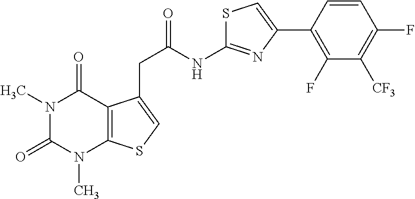 [SMILES: Cn1c(=O)c2c(CC(=O)Nc3nc(-c4ccc(F)c(C(F)(F)F)c4F)cs3)csc2n(C)c1=O]
a beta-2 agonist indacaterol or its salt and a pharmaceutically acceptable excipient.

59. The pharmaceutical composition according to claim 58, wherein the composition is a fixed dose combination.

60. The pharmaceutical composition according to claim 58, wherein the composition is for oral administration and the TRPA1 antagonist and the beta-2 agonist are present in a weight ratio from about 1:0.01 to about 1:5.

61. A method of treating a respiratory disorder in a subject, said method comprising administering to the subject the pharmaceutical composition according to claim 58.

62. A method of improving lung function in a subject, said method comprising administering to the subject the pharmaceutical composition according to claim 58.

63. The method according to claim 61, wherein the respiratory disorder is chronic obstructive pulmonary disease.

64. A pharmaceutical composition comprising synergistically effective amount of a TRPA1 antagonist having structure: 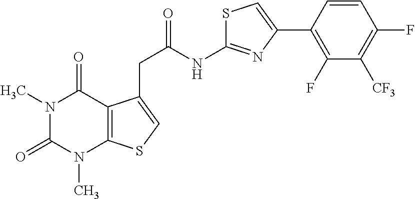 [SMILES: Cn1c(=O)c2c(CC(=O)Nc3nc(-c4ccc(F)c(C(F)(F)F)c4F)cs3)csc2n(C)c1=O]
a beta-2 agonist olodaterol or its salt and a pharmaceutically acceptable excipient.

65. The pharmaceutical composition according to claim 64, wherein the composition is a fixed dose combination.

66. The pharmaceutical composition according to claim 64, wherein the composition is for oral administration and the TRPA1 antagonist and the beta-2 agonist are present in a weight ratio from about 1:0.01 to about 1:5.

67. A method of treating a respiratory disorder in a subject, said method comprising administering to the subject the pharmaceutical composition according to claim 64.

68. A method of improving lung function in a subject, said method comprising administering to the subject the pharmaceutical composition according to claim 64.

69. The method according to claim 67, wherein the respiratory disorder is chronic obstructive pulmonary disease.

70. A pharmaceutical composition comprising synergistically effective amount of a TRPA1 antagonist having structure: 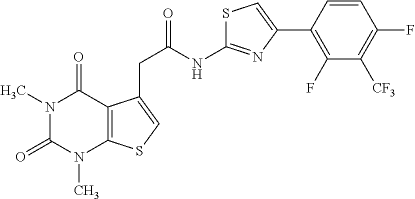 [SMILES: Cn1c(=O)c2c(CC(=O)Nc3nc(-c4ccc(F)c(C(F)(F)F)c4F)cs3)csc2n(C)c1=O]
a beta-2 agonist vilanterol or its salt and a pharmaceutically acceptable excipient.

71. The pharmaceutical composition according to claim 70, wherein the composition is a fixed dose combination.

72. The pharmaceutical composition according to claim 70, wherein the composition is for oral administration and the TRPA1 antagonist and the beta-2 agonist are present in a weight ratio from about 1:0.01 to about 1:5.

73. A method of treating a respiratory disorder in a subject, said method comprising administering to the subject the pharmaceutical composition according to claim 70.

74. A method of improving lung function in a subject, said method comprising administering to the subject the pharmaceutical composition according to claim 70.

75. The method according to claim 73, wherein the respiratory disorder is chronic obstructive pulmonary disease.
